(19) United States
(12) Patent Application Publication
(10) Pub. No.: US 2003/0181498 A1
(43) Pub. Date: Sep. 25, 2003

(54) Bicyclic heteroaryl compounds as inhibitors of the interaction between the integrin alpha4beta1 receptor and vcam-1 and/or fibronectin

(75) Inventors: David Robert Brittain, Macclesfield (GB); Michael Stewart Large, Macclesfield (GB); Gareth Morse Davies, Macclesfield (GB)

Correspondence Address: ROPES & GRAY LLP, ONE INTERNATIONAL PLACE, BOSTON, MA, 02110-2624, US

(73) Assignee: AstraZeneca AB, Sodertalje (SE)

(21) Appl. No.: 10/168,396

(22) PCT Filed: Jan. 17, 2001
(86) PCT No.: PCT/GB01/00161

(30) Foreign Application Priority Data
Jan. 21, 2000 (GB) 0001346.6

(51) Int. Cl. C07D 263/52; A61K 31/423

(52) U.S. Cl. 514/375; 548/217

(57) ABSTRACT

A compound of formual (I) or pharmaceutically acceptable salts or derivatives thereof; wherein variables are as defined in the specification. The compounds are useful in the treatment of disease mediated by the interaction between VCAM-1 and/or fibronectin and the integrin receptor α₄β₁. Pharmaceutical compositions and methods of use or treatment are also described and claimed.

This invention relates to compounds which are inhibitors of the interaction between the integrin α4β₁, also known as Very Late Antigens (VLA-4) or CD49d/CD29, and its protein ligands, for example Vascular Cell Adhesion Molecule-1 (VCAM-1) and fibronectin. This invention further relates to processes for preparing such compounds, to pharmaceutical compositions containing them and to their use in methods of therapeutic application.

α₄β₁ is a member of the integrin family of heterodimeric cell surface receptors that are composed of noncovalently associated glycoprotein subunits (α and β) and are involved in cell adhesion to other cells or to extracellular matrix. There are at least 14 different human integrin a subunits and at least 8 different β subunits and each β subunit can form a heterodimer with one or more α subunits. Integrins can be subdivided based on their β subunit composition. α₄β₁ is one of several β₁ integrins, also known as Very Late Antigens (VLA).

The interactions between integrins and their protein ligands are fundamental for maintaining cell function, for example by tethering cells at a particular location, facilitating cell migration, or providing survival signals to cells from their environment Ligands recognised by integrins include extracellular matrix proteins, such as collagen and fibronectin; plasma proteins, such as fibrinogen; and cell surface molecules, such as transmembrane proteins of the immunoglobulin superfamily and cell-bound complement. The specificity of the interaction between integrin and ligand is governed by the α and β subunit composition.

Integrin α₄β₁ is expressed on numerous hematopoietic cells and established cell lines, including hematopoietic precursors, peripheral and cytotoxic T lymphocytes, B lymphocytes, monocytes, thymocytes and eosinophils [Hemler, M. E. et al (1987), J. Biol. Chem., 262, 11478-11485; Bochner, B. S. et al (1991), J. Exp. Med., 173, 1553-1556]. Unlike other β₁ integrins that bind only to cell-extracellular matrix proteins, α₄β₁ binds to VCAM-1, an immunoglobulin superfamily member expressed on the cell surface, for example on vascular endothelial cells, and to fibronectin containing the alternatively spliced type III connecting segment (CS-1 fibronectin) [Elices, M. J. et al (1990), Cell, 60, 577-584; Wayner, E. A. et al (1989). J. Cell Biol., 109, 1321-1330].

The activation and extravasation of blood leukocytes plays a major role in the development and progression of inflammatory diseases. Cell adhesion to the vascular endothelium is required before cells migrate from the blood into inflamed tissue and is mediated by specific interactions between cell adhesion molecules on the surface of vascular endothelial cells and circulating leukocytes [Sharar, S. R. et al (1995). Springer Semin. Immunopathol., 16, 359-378]. α₄β₁ is believed to have an important role in the recruitment of lymphocytes, monocytes and eosinophils during inflammation. α₄β₁/ligand binding has also been implicated in T-cell proliferation, B-cell localisation to germinal centres, haemopoeitic progenitor cell localisation in the bone marrow, placental development, muscle development and tumour cell metastasis.

The affinity of α₄β₁ for its ligands is normally low but chemokines expressed by inflamed vascular endothelium act via receptors on the leukocyte surface to upregulate α₄β₁ function [Weber, C. et al (1996), J. Cell Biol., 134, 1063-1073]. VCAM-1 expression is upregulated on endothelial cells in vitro by inflammatory cytokines [Osborn, L. et al (1989) Cell, 59, 1203-1211] and in human inflammatory diseases such as rheumatoid arthritis [Morales-Ducret, J. et al (1992). J. Immunol., 149, 1424-1431], multiple sclerosis [Cannella, B. et al., (1995). Ann. Neurol., 37, 424435], allergic asthma [Fukuda, T. et al (1996), Am. J. Respir. Cell Mol. Biol., 14, 84-94] and atherosclerosis [O'Brien, K. D. et al (1993). J. Clin. Invest., 92, 945-951].

Monoclonal antibodies directed against the α₄ integrin subunit have been shown to be effective in a number of animal models of human inflammatory diseases including multiple sclerosis, rheumatoid arthritis, allergic asthma, contact dermatitis, transplant rejection, insulin-dependent diabetes, inflammatory bowel disease, and glomerulonephritis.

Integrins recognise short peptide motifs in their ligands The minimal α₄β₁ binding epitope in CS-1 is the tripeptide leucine-aspartic acid-valine (Leu-Asp-Val) [Komoriya, A, et al (1991). J. Biol. Chem., 266, 15075-15079] while VCAM-1 contains the similar sequence isoleucine-aspartic acid-serine [Clements, J. M., et al (1994). J. Cell Sci., 107, 2127-2135]. The 25-amino acid fibronectin fragment, CS-1 peptide, which contains the Leu Asp-Val motif, is a competitive inhibitor of α₄β₁ binding to VCAM-1 [Makarem, R., et al (1994). J. Biol. Chem., 269, 4005-4011]. Small molecule α₄β₁ inhibitors based on the Leu-Asp-Val sequence in CS-1 have been described, for example the linear molecule phenylacetic acid-Leu-Asp-Phe-D-Pro-amide [Molossi, S. et al (1995). J. Clin. Invest., 95, 2601-2610] and the disulphide cyclic peptide Cys-Trp-Leu-Asp-Val-Cys [Vanderslice, P., et al (1997). J. Immunol., 158, 1710-1718].

More recently, non- and semi-peptidic compounds which inhibit α₄β₁/VCAM binding and which can be orally administered have been reported in for example, WO96/22966 and WO98/04247.

There remains a continuing need for alternative compounds which inhibit the interaction between VCAM-1 and fibronectin with integrin α₄β₁ and, in particular, for compounds which can be administered by an oral route.

Our copending International Patent Application No PCT/GB99/02330 describes a series of compounds which contain a bicyclic heterocylic ring which inhibit this interaction. Further compounds which have this effect have now been found.

Accordingly the present invention provides a compound of formula (I)

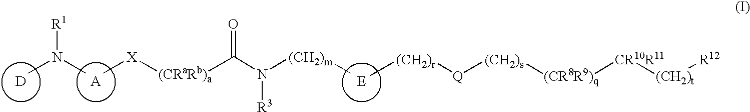

wherein:

A is a bicyclic heteroaryl group, optionally substituted with one or more substituents independently selected from C₁₋₄ alkyl, C₁alkanoyl, C₂₋₆alkenyl, C₂₋₆alkynyl, C₁₋₆alkoxy, C₁₋₆ alkylamino, C₁₋₆alkylthio, C₁₋₄ alkylsulphonyl, C₁₋₄alkoxylC₁₋₆alkyl, C₁₋₆alkylaminoC₁₋₆alkyl, carboxy, carbamoyl, C₂₋₆alkenyloxy, C₂₋₆alkynyloxy, di-[(C₁₋₆)alkyl]amino, C₂₋₆alkanoylamino, N—C₁₋₆alkycarbamoyl, C₁₋₆alkoxylcarbonyl, halogeno, nitro, cyano, amino trifluoromethyl, trifluoromethoxy, hydroxy, (CH₂)ₚOH where p is 1 or 2, —CO₂R^{e1}, and —CONR^{e1}R^{f1}, where R^{e1} and R^{f1} are independently hydrogen or C₁₋₆ alkyl; and linked to the nitrogen via a ring carbon atom in one ring and to the group Z by a ring carbon atom in the second ring;

D is aryl or a mono or bicyclic heteroaryl group, each of which can be optionally subsitituted with one or more substituents independently selected from C₁₋₆ alkyl, C₂₋₆alkenyl, C₂₋₆alkynyl, C₁₋₆ alkoxy, C₁₋₄ alkanoyl, C₁₋₆ alkylamino, C₁₋₆ alkylthio, C₁₋₄ alkylsulphonyl, C₁₋₄alkoxylC₁₋₆alkyl, C₁₋₆alkylaminoC₁₋₆alkyl, carboxy, carbamoyl, C₂₋₆ alkenyloxy, C₂₋₆alkynyloxy, di-[(C₁₋₆)alkyl]amino, C₂₋₆alkanoylamino, N—C₁₋₆alkylcarbamoyl, C₁₋₆alkoxylcarbonyl, phenoxy, cyano, nitro, amino, halogeno, trifluoromethyl, trifluoromethoxy, hydroxy, (CH₂)ₚOH where p is 1 or 2, —CO₂R^{e2}, and —CONR^{e2}R^{f2}, where R^{e2} and R^{f2} are independently hydrogen or C₁₋₆ alkyl, or two adjacent substitutents on the group D together with the ring atoms to which they are attached, form a 5-7membered optionally substituted ring which may contain up to three heteroatoms, and D is linked to NR¹ through a ring carbon atom;

R^{a} and R^{b} are independently hydrogen or C₁₋₄ alkyl;

a is an integer from 1 to 4;

X is a direct bond, oxygen, sulphur, amino or C₁₋₄alkylamino;

R¹ is hydrogen, C₁₋₅ alkyl, C₁₋₃ alkanoyl or C₁₋₃ alkoxycarbonyl;

R³ is hydrogen or C₁₋₅ alkyl;

E is a monocyclic or bicyclic heterocyclic ring containing at least one linking nitrogen atom, and which is optionally substituted with one or more substituents independently selected from oxo, C₁₋₆ alkyl, C₂₋₆alkenyl, C₂₋₆alkynyl, C₁₋₆ alkoxy, C₁₋₄ alkanoyl, C₁alkylamino, C₁₋₄alkoxylC₁₋₆alkyl, C₁₋₆alkylaminoC₁₋₆alkyl, nitro, cyano, halogeno, trifluoromethyl, trifluoromethoxy, hydroxy, (CH₂)ₚOH where p is 1 or 2, —CO₂R^{e3}, and —CONR^{e3}R^{f3}, where R^{e3} and R^{f3} are independently selected from hydrogen and C₁₋₆ alkyl; and a substituent of formula (V)

—U—(CH₂)_{d}V-T (V)

wherein U is selected from oxygen, sulphur, a direct bond or —CH₂O—, V is selected from nitrogen, oxygen, sulphur or a direct bond, d is zero or a number from 1 to 4, and T is selected from R^{c} or, when V is nitrogen, R^{c}R^{d}, where R^{c} and R^{d} are independently selected from hydrogen, C₁₋₄ C₁₋₄ alkoxy, C₁₋₄ alkoxy(C₁₋₆)alkyl or aryl; or T is a heterocycle containing up to three heteroatoms selected from nitrogen, oxygen and sulphur, optionally substituted with one or more substituents selected from C₁₋₆ alkyl, C₂₋₆alkenyl, C₂₋₆alkynyl, C₁₋₆alkoxy, C₁₋₄ alkanoyl, C₁₋₆ alkylamino, C₁₋₄alkoxylC₁₋₆alkyl, C₁₋₆alkylaminoC₁₋₆alkyl, C₁₋₄ alkylsulphonyl, nitro, cyano, halogeno, trifluoromethyl, trifluoromethoxy, hydroxy, (CH₂)ₚOH where p is 1 or 2, —CO₂R^{e4}, and —CONR^{e4}R^{f4}, where R^{e4} and R^{f4} are independently selected from hydrogen and C₁₋₆ alkyl, and linked to V through a ring carbon or nitrogen and with the proviso that when T is a heterocycle linked to V through a ring nitrogen then V is a direct bond;

Q is selected from a direct bond, methylene, oxygen, carbonyl, —C(OH)(H)—, C₂ alkenyl or C₂ alkynyl;

R¹⁰ and each R⁸ and R⁹ are independently selected from hydrogen, C₁₋₆ alkyl, aryl and heterocycle, the aryl and heterocycle being optionally substituted with one or more substituents independently selected from C₁₋₆ alkyl, C₂₋₆alkenyl, C₁₋₄alkanoyl, C₂₋₆alkynyl, C₁₋₆ alkoxy, C₁₋₆ alkylamino, C₁₋₄alkylC₁₋₆alkyoxyl, C₁₋₆alkylaminoC₁₋₆allyl nitro, cyano, halogeno, trifluoromethyl, hydroxy, (CH₂)ₚOH where p is 1 or 2, —CO₂R^{e5}, and —CONR^{e5}R^{f5}, where R^{e5} and R^{f5} are independently selected from hydrogen and C₁₋₆ alkyl, or two of R⁸, R⁹ and R¹⁰ together form a phenyl or a 3-7 membered heterocycle; R¹¹ is selected from hydrogen, C₁₋₆ alkyl, C₂₋₆alkenyl, 1,3-benzodioxol-5-yl, an ester group, hydroxy, amido, heterocycle and aryl, the heterocycle, and aryl optionally substituted with one or more substituents independently selected from C₁₋₆ alkyl, C₂₋₆alkenyl, C₁₋₄alkanoyl, C₂₋₆alkynyl, C₁₋₆ alkoxy, C₁₋₆ alkylamino, C₁₋₄alkylC₁₋₆alkyoxyl, C₁₋₆alkylaminoC₁₋₆alkyl, nitro, cyano, halogeno, trifluoromethyl, hydroxy, (CH₂)ₚOH where p is 1 or 2, —CO₂R^{e6}, —CONR^{e6}R^{f6}, where R^{e6} and R^{f6} are independently selected from hydrogen and C₁₋₆ alkyl,

R¹² is an acidic functional group;

r is zero or 1;

q is 0, 1 or 2;

s is zero, 1 or 2;

t is zero or an integer of from 1 to 3;

m is zero or an integer of from 1 to 3;

or a pharmaceutically acceptable salt or in vivo hydrolysable derivative thereof.

In this specification the following definitions are adopted:—

The term ‘heterocycle’ includes an aromatic or non-aromatic saturated or partially unsaturated cyclic ring systems containing up to five heteroatoms independently selected from nitrogen, oxygen and sulphur. Suitably heterocycles will contain up to 20 and preferably up to 12 atoms in total. Heterocycles with two or more rings may include a mixture of aromatic and non-aromatic rings, or they may be completely aromatic or completely non-aromatic.

Unless otherwise stated, suitable optional substituents for heterocycles include one or more substituents selected from oxo, C₁₋₆ alkyl, C₂₋₆alkenyl, C₂₋₆alkynyl, C₁₋₆ alkoxy, C₁₋₄ alkanoyl, C₁₋₆ alkylamino, C₁₋₄alkoxylC₁₋₆alkyl, C₁₋₆alkylaminoC₁₋₆alkyl, C₁₋₄ alkylsulphonyl, nitro, cyano, halogeno, trifluoromethyl, trifluoromethoxy, hydroxy, (CH₂)ₚOH where p is 1 or 2, —CO₂R^{e}, and —CONR^{e}R^{f}, where R^{e} and R^{f} are independently selected from hydrogen and C₁₋₆ alkyl. Examples include 3 to 10 membered monocyclic or bicyclic rings with up to five heteroatoms selected from oxygen, nitrogen and sulphur, such as, for example, furanyl, pyrrolinyl, piperidinyl, piperazinyl, thienyl, pyridyl, imidazolyl, tetrazolyl, thiazolyl, pyrazolyl, pyrimidinyl, triazinyl, pyridazinyl, pyrazinyl, morpholinyl, oxiranyl, oxetanyl, tetrahydrofuranyl, pyrrolidinyl, imidazolinyl, imidazolidinyl, pyrazolinyl, pyrazolidinyl, piperidinyl, homopiperazinyl, dihydropyridinyl, tetrahydropyridinyl, dihydropyrimidinyl and tetrahydropyrimidinyl.

The monocyclic heteroaryl is a aromatic ring system containing up to four heteroatoms, examples of which are given above.

‘Bicyclic heteroaryl’ means an aromatic 5,6-6,5- or 6,6-fused ring system wherein one or both rings contain ring heteroatoms. The ring system may contain up to three heteroatoms, independently selected from oxygen, nitrogen or sulphur. Particular optional substitutents for such bicyclic heteroaryl groups are one or more substituents selected from C₁₋₆ alkyl, C₂₋₆ alkenyl, C₂₋₆alkynyl, C₁₋₆ alkoxy, C₁₋₄ alkanoyl, C₁₋₆ alkylamino, C₁₋₄alkoxylC₁₋₆alkyl, C₁₋₆alkylaminoC₁₋₆alkyl, C₁₋₄ alkylsulphonyl, nitro, cyano, halogeno, trifluoromethyl, trifluoromethoxy, hydroxy, (CH₂)ₚOH where p is 1 or 2, —CO₂R^{e8}, and —CONR^{e8}R^{f8}, where R^{e8} and R^{f8} are independently selected from hydrogen and C₁₋₆ alkyl. When the ring system contains more than one heteratom at least one heteroatom is nitrogen Examples of bicyclic heteroaryl's include quinazolinyl, benzothiophenyl, benzoxazolyl, benzimidazolyl, benzothiazolyl, benzofuranyl, indolyl, quinolinyl, phthalazinyl and benzotriazolyl.

‘Aryl’ typically means phenyl or naphthyl, preferably phenyl.

The 5 to 7 membered ring formed by substituents on ring D or substituents R¹³, see below, can be an, optionally substituted, saturated or unsaturated ring with up to three heteroatoms independently selected from nitrogen, oxygen and sulphur. Suitable substituents include those listed above in relation to heterocycles.

D is suitably an aryl or a mono or bicyclic heteroaryl group, each of which can be optionally subsitituted with one or more substituents independently selected from C₁₋₆ alkyl, C₂₋₆alkenyl, C₂₋₆alkynyl, C₁₋₆ alkoxy, C₁₋₄ alkanoyl, C₁₋₆ alkylamino, C₁₋₆ alkylthio, C₁₋₄ alkylsulphonyl, C₁₋₄alkoxylC₁₋₆alkyl, C₁₋₆alkylaminoC₁₋₆alkyl, carboxy, carbamoyl, C₂₋₆ alkenyloxy, C₂₋₆alkynyloxy, di-[(C¹⁻⁶)alkyl]amino, C₂₋₆alkanoylamino, N—C₁₋₆alkylcarbamoyl, C₁₋₆alkoxylcarbonyl, phenoxy, cyano, nitro, amino, halogeno, trifluoromethyl, trifluoromethoxy, hydroxy, (CH₂)ₚOH where p is 1 or 2, —CO₂R^{e2}, and —CONR^{e2}R^{f2}, where R^{e2} and R^{f2} are as defined above.

Preferably, ring D is unsubstituted.

The term ‘acidic functional group’ means a group which incorporates an acidic hydrogen and includes carboxylic acids, tetrazoles, acyl sulphonamides, sulphonic and sulphinic acids, and preferably is carboxy.

The term “alkyl” as used herein, will generally include straight or branched C₁₋₆alkyl unless stated otherwise.

The term ‘ester group’ is an ester derived from a C₁₋₁₀ straight or branched allyl, arylalkyl or C₅₋₇ cycloalkyl (optionally substituted with C₁₋₄ alkyl) alcohol. Suitable ester groups are those of formula —COOR″ where R″ can be tert-butyl, 2,4-dimethyl-pent-3-yl, 4-methyl-tetrahydropyran-4-yl, 2,2-dimethyl aminoethyl or 2-methyl 3-phenyl prop-2-yl.

In this specification suitable specific groups for the substituents mentioned

| | |
|---|---|
| for halogeno: | fluoro, chloro, bromo and iodo |
| for C₁₋₆alkyl (this | methyl, ethyl, propyl, isopropyl, tert- |
| includes straight chained, | butyl, cyclopropane and cyclohexane; |
| branched structures | |
| and ring systems): | |
| for C₂₋₆alkenyl: | vinyl, allyl and but-2-enyl; |
| for C₁₋₆alkanoyl; | formyl, acetyl, propionyl or butyryl; |
| for C₂₋₆alkynyl: | ethynyl, 2-propynyl and but-2-ynyl; |
| for C₁₋₆alkoxy: | methoxy, ethoxy, propoxy, isopropoxy |
| | and butoxy; |
| for C₂₋₆alkenyloxy: | vinyloxy and allyloxy; |
| for C₂₋₆alkynyloxy: | ethynyloxy and 2-propynyloxy; |
| for C₁₋₆alkylamino: | methylamino, ethylamino, propylamino, |
| | isopropylamino and butylamino; |
| for di-C₁₋₆alkylamino: | dimethylamino, diethylamino; |
| for C₂₋₆alkanoylamino: | acetamido, propionamido and butyramido; |
| for N-C₁₋₆alkylcarbamoyl: | N-methylcarbamoyl, N-ethylcarbamoyl |
| | and N-propylcarbamoyl; |
| for C₁₋₆alkoxycarbonyl: | methoxycarbonyl, ethoxycarbonyl, |
| | propoxycarbonyl and tert-butoxycarbonyl; |
| for C₁₋₄alkoxyC₁₋₆alkyl: | methoxymethyl, ethoxymethyl, |
| | 1-methoxymethyl, 2-methoxyethyl; |
| for C₁₋₆alkylthio: | methylthio; |
| For C₁₋₄alkylsulphonyl: | methylsulphonyl; |
| For C₁₋₆alkylaminoC₁₋₆alkyl: | —CH₂NHC₂H₅ |

It is to be understood that, insofar as certain of the compounds of the formula (I), defined above and below may exist in optically active or racemic forms by virtue of one or more asymmetric carbon atoms, the invention encompasses any such optically active or racemic form which can inhibit the interaction between VCAM-1 and fibronectin with the integrin α₄β₁. The synthesis of optically active forms may be carried out by standard techniques of organic chemistry well known in the art, for example by synthesis from optically active starting materials or by resolution of a racemic form.

Suitably in the compound of formula (I), D is a phenyl optionally substituted with up to five substituents independently selected from C₁₋₆ alkyl, C₂₋₆alkenyl, C₂₋₆alkynyl C₁₋₄ alkoxy, C₁₋₄ alkanoyl, C₁₋₆ alkylamino, C₁₋₄alkoxylC₁₋₆alkyl, C₁₋₆allylaminoC₁₋₆alkyl, cyano, nitro, halogeno, trifluoromethyl, hydroxy, (CH₂)ₚOH where p is 1 or 2, are —CO₂R^{e}, and —CONR^{e2}R^{f2}, where R^{e2} and R^{f2} are independently hydrogen and C₁₋₆ alkyl, or two adjacent substituents can be taken together to form a 5-7 membered ring.

Thus, in a further aspect of the invention the compound has the formula (II)

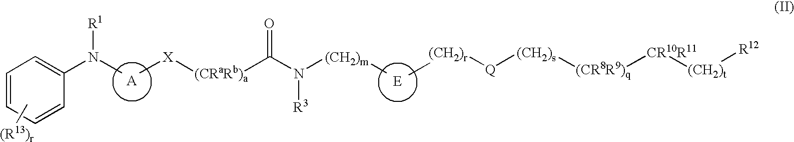

wherein:

A, R¹, X, R^{a}, R^{b}, a, R³, E, m, r, Q, s, R⁸, R⁹, q, R¹⁰, R¹¹, t and and R¹² are as hereinbefore defined;

each R¹³ is independently selected from C₁₋₆ alkyl, C₂₋₆alkenyl, C₂₋₆alkynyl, C₁₋₄ alkoxy, C₁₋₄ alkanoyl, C₁₋₆ alkylamino, C₁₋₄alkoxylC₁₋₆alkyl, C₁₋₆alkylaminoC₁₋₆alkyl, cyano, nitro, halogeno, trifluoromethyl, hydroxy, (CH₂)ₚOH where p is 1 or 2, —CO₂R^{e}, and —CONR^{e}R^{f}, where R^{e} and R^{f} are independently hydrogen and C₁₋₆ alkyl, or where f is at least 2, two adjacent groups R¹³ can be taken together to form a 5-7 membered ring; and

f is zero or an integer from 1 to 5.

In another preferred embodiment, t is 0 and q is 2, where at least one pair of R⁸ and R⁹ are both hydrogen. In particular, a group of sub-formula (v)

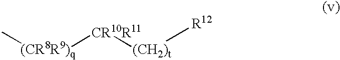

as found in formula (I) is a group of subformula (vi):

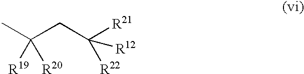

where R¹² is as defined in relation to formula ([) and R¹⁹ to R²² are each independently selected from hydrogen, C₁₋₄ alkyl, aryl and heteroaryl containing up to 2 heteroatoms chosen from oxygen, sulphur and nitrogen, the aryl and heteroaryl optionally substituted with one or more substituents selected from nitro, C¹⁻⁶ alkyl, C₂₋₆alkenyl, C₂₋₆alkynyl, C₁₋₄alkoxy, C₁₋₄ alkylamino, C₁₋₄alkylC₁₋₆alkyoxyl, C₁₋₆alkylaminoC₁₋₆alkyl, cyano, halogeno, trifluoromethyl, hydroxy, (CH₂)ₚOH where p is 1 or 2, —CO₂R^{e}, and —CONR^{e7}R^{f7}, where R^{e7} and R^{f7} are independently selected from hydrogen and C₁₋₆alkyl or two of R¹⁹, R²⁰ or R²¹ can together form a phenyl or 3 to 7 membered heterocycle.

In particular, the compound of formula (II) is s compound of formula (III)

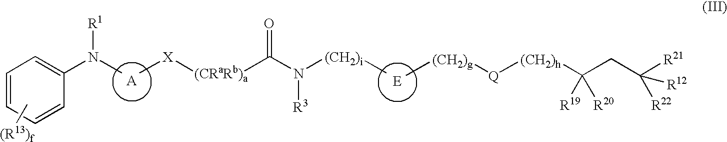

where A, R¹, Q, X, R^{a}, R^{b}, a, R³, E, R¹² and R¹³ and f are as hereinbefore defined in relation to formula (II) and R¹⁹ to R²² are as defined above in relation to sub-formula (vi),

and g, h and i are each independently 0 or 1;

or a pharmaceutically acceptable salt or in vivo hydrolysable derivative thereof.

The ring E may be linked either to the —NR³(CH₂)ₘ— group or to the —(CH₂)_{g}Q-group or to both of these groups by way of a nitrogen atom, provided only that when it is linked to the NR³(CH₂)ₘ— group by way of a nitrogen atom, m is at least 1, and when it is linked to the —(CH₂)_{g}Q-group by way of a nitrogen atom, g is at least 1. Preferably, the ring E is linked to the —(CH)_{g}Q-group by way of a nitrogen atom,

The ring E is suitably a monocyclic or bicyclic heterocycle containing at least one and suitably from 1 to 3 nitrogen atoms. It may further contain additional heteroatoms selected from oxygen or sulphur. Where the ring contains sulphur, this may be oxidised to S(O) or S(O). Rings may be aromatic, non-aromatic or, in the case of bicyclic rings, mixed as described above. Preferably, the ring E is heteroaryl.

Preferably, E is a monocyclic heterocyclic ring preferably of 5 or 6 atoms, up to 3 of which are nitrogen atoms. Suitably the ring contains 1 or 2 nitrogen atoms. They may be aromatic or non-aromatic such as N-linked tetrahydropyridyl, but are preferably aromatic. Examples of E include N-linked pyridone, pyrimidone, triazole, imidazole pyrazole, or pyrrole group, and in particular, N-linked pyridone, pyrimidone, imidazole or pyrazole.

Particular substituents for group E include one or more groups selected from C₁₋₆ alkyl, C₂₋₆alkenyl, C₂₋₆alkynyl, C₁₋₄ alkoxy, C₁₋₄ alkanoyl, C₁₋₆ alkylamino, C₁₋₄alkoxylC₁₋₆alkyl, C₁₋₆alkylaminoC₁₋₆alkyl, nitro, cyano, halogeno, trifluoromethyl, hydroxy, (CH₂)ₚOH where p is 1 or 2 —CO₂R^{e3}, and —CONR^{e3}R^{f3}, where R^{e3} and R^{f3} are as defined above, or a group of formula (V) as defined above.

Particular examples of aromatic rings E are rings of sub-formula (i), (ii), (iii) or (iv)

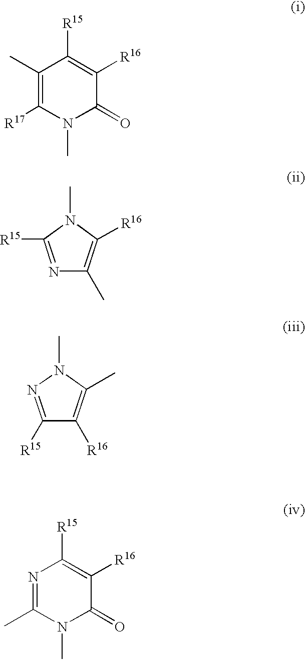

where R¹⁵ to R¹⁷ are each independently hydrogen, C₁₋₆ alkyl, C₂₋₆alkenyl, C₂₋₆alkynyl, C₁₋₄ alkoxy, C₁₋₄ alkanoyl, C₁₋₆ alkylamino, C₁₋₄alkoxylC₁₋₆alkyl, C¹⁻⁶alkylaminoC₁₋₆alkyl, nitro, cyano, halogeno, trifluoromethyl hydroxy, (CH₂)ₚOH were p is 1 or 2 —CO₂R^{e3}, and —CONR^{e3}R^{f3}, where R^{e3} and R^{f3} are as defined above in relation to formula (I) or a substitutent of formula (V) as defined above.

Preferably in groups of sub-formula (i)-(iv), R¹⁵, R¹⁶ and R¹⁷ are all hydrogen.

Suitably in the compounds of formula (I), R^{a} and R^{b} are both hydrogen

Preferably a is 1.

A preferred group A is benzoxazolyl.

Thus in a preferred sub-group of compounds of formula (I), R^{a} and R^{b} are both hydrogen, a is 1, and A is benzoxazolyl, optionally substituted as hereinbefore defined. Thus particular examples of compounds of formula (1) included compounds of formula (IV)

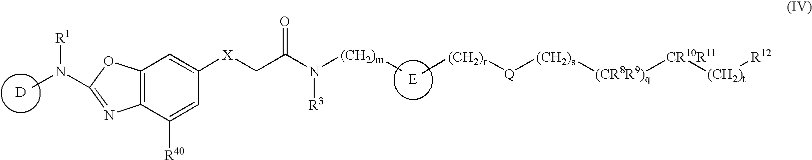

where

D, R¹, X, R³, E, Q, R⁸, R⁹, R¹⁰, R¹¹, R¹², m, r, s, q and t are as defined above, and R⁴⁰ is hydrogen, C₁₋₄ alkoxy, halogeno, alkylthio and alkylsulphonyl, and especially, for example hydrogen or methoxy.

Preferably, X is a direct bond or oxygen, and most preferably a direct bond.

Preferably R¹ is hydrogen or C₁₋₂ alkyl, more preferably hydrogen.

Preferably R³ is hydrogen or C₁₋₂ alkyl, more preferably hydrogen.

Preferably, m, r and s are equivalent to i, g and h respectively.

Preferably Q is a direct bond or oxygen and is preferably a direct bond.

Most preferably R¹² is carboxy.

Preferably R⁸, R⁹, R¹⁰ and R¹¹ are selected from hydrogen or C₁₋₄alkyl such as methyl, and most preferably, they are hydrogen.

Suitably r+s+q+t are equal to 0 or an integer of 1 or 2.

Particularly compounds of formula (I) are those described in the Examples and in Table 1.

TABLE 1

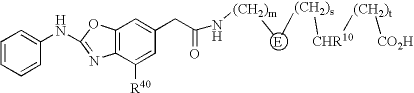

| Compd No. | m | E | s | R¹⁰ | t | R⁴⁰ |
|---|---|---|---|---|---|---|
| CI | 0 | 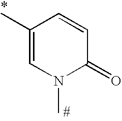 | 1 | H | 1 | H |
| CII | 0 | 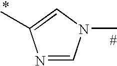 | 1 | H | 1 | H |
| CIII | 0 | 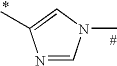 | 1 | H | 2 | H |
| CIV | 0 | 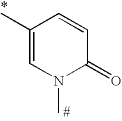 | 1 | H | 2 | H |
| CV | 0 | 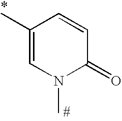 | 1 | H | 2 | OCH₃ |
| CVI | 0 | 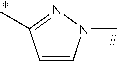 | 1 | H | 1 | H |
| CVII | 0 | 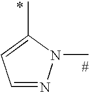 | 1 | H | 1 | H |
| CVIII | 0 | 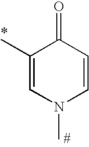 | 1 | H | 1 | H |
| CIX | 0 | 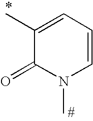 | 0 | H | 0 | H |
| CX | 0 | 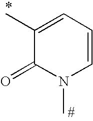 | 0 | H | 1 | H |
| CXI | 0 | 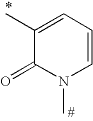 | 1 | H | 1 | H |
| CXII | 0 | 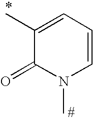 | 1 | CH₃ | 1 | H |
| CXIII | 0 | 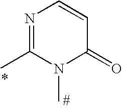 | 1 | H | 1 | H |
| CXIV | 3 | 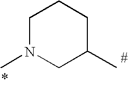 | 0 | H | 0 | H |
| CXV | 1 | 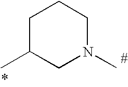 | 0 | H | 0 | H |
| CXVI | 1 | 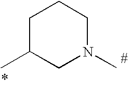 | 1 | H | 1 | OCH₃ |
| CXVII | 0 | 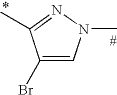 | 1 | H | 1 | H |
| CXVIII | 0 | 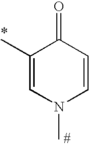 | 1 | H | 1 | OCH₃ |

In the above definition of E, * indicates the point of attachment to the group —NH(CH₂)ₘ and # indicates the point of attachment to the group —(CH₂)_{S}CHR¹⁰—

Pharmaceutically acceptable salts include acid addition salts such as salts formed with mineral acids, for example, hydrogen halides such as hydrogen chloride and hydrogen bromide, sulphonic and phosphonic acids; and salts formed with organic acids, especially citric, maleic, acetic, oxalic, tartaric, mandelic, p-toluenesulphonic, methanesulphonic acids and the like. In another aspect, suitable salts are base salts such as alkali metals salts, for example, sodium and potassium; alkaline earth metal salts such as magnesium and calcium; aluminium and ammonium salts; and salts with organic bases such as ethanolamine, methylamine, diethylamine, isopropylamine, trimethylamine and the like. Such salts may be prepared by any suitable method known in the art.

In vivo hydrolysable derivatives include, in particular, pharmaceutically acceptable derivatives that may be oxidised or reduced in the human body to produce the parent compound or esters that hydrolyse in hte human body to produce the parent compound. Such esters can be identified by administering, for example, intravenously to the test animal, the compound under test and subsequently examining the test animal's body fluids. Suitable in vivo hydrolysable esters for hydroxy include acetyl and for carboxyl include, for example, alkyl esters, dialkylaminoalkoxy esters, esters of formula —C(O)—O—CH₂C(O)NR^{a}″R^{b}″ where R^{a}″ and R^{b}″ are, for example, selected from hydrogen and C₁₋₄ alkyl, and C₁₋₆alkoxy methyl esters for example methoxymethyl, C₁₋₆alkanoyloxymethyl esters for example pivaloyloxymethyl, phthalidyl esters, C₃₋₈ cycloalkoxycarbonyloxyC₁₋₆alkyl esters for example 1-cyclohexylcarbonyloxyethyl; 1,3-dioxolan-2-ylmethyl esters for example 5-methyl-1,3-dioxolan-2-ylmethyl; and C₁₋₆alkoxycarbonyloxyethyl esters for example 1-methoxycarbonyloxyethyl.

The activities of the compounds of this invention to inhibit the interaction between VCAM-1 and fibronectin with integrin α₄β₁ may be determined using a number of in vitro and in vivo screens.

For example, compounds of formulae (I), (II), (III) or (IV) preferably have an IC₅₀ of <10 μM, more preferably <1 μM in the MOLT4 cell/Fibronectin assay hereinafter described.

In order for it to be used, a compound of formulae (I), (II), (III) or (IV) or a pharmaceutically acceptable salt or an in vivo hydrolysable derivative thereof is typically formulated as a pharmaceutical composition in accordance with standard pharmaceutical practice.

Thus, according to a further aspect of the invention there is provided a pharmaceutical composition which comprises a compound of formulae (I), (II), (III) or (IV) or a pharmaceutically acceptable salt or an in vivo hydrolysable derivative thereof and a pharmaceutically acceptable carrier.

The pharmaceutical compositions of this invention may be in a form suitable for oral use, for example a tablet, capsule, aqueous or oily solution, suspension or emulsion; for nasal use, for example a snuff, nasal spray or nasal drops; for vaginal or rectal use, for example a suppository; for administration by inhalation, for example as a finely divided powder or a liquid aerosol; for sub-lingual or buccal use, for example a tablet or capsule; or for parenteral use (including intravenous, subcutaneous, intramuscular, intravascular or infusion), for example a sterile aqueous or oily solution or suspension, or a depot formulation with drug incorporated in a biodegradable polymer. The composition may be in a form suitable for topical administration such as for example creams, ointments and gels. Skin patches are also contemplated. For these purposes, the compositions of this invention may be formulated by means known in the art, such as for example, as described in general terms, in Chapter 25.2 of Comprehensive Medicinal Chemistry, Volume 5, Editor Hansch et al, Pergamon Press 1990.

Furthermore, the pharmaceutical composition of the present invention may contain one or more additional pharmacological agents suitable for treating one or more disease conditions referred to hereinabove, in addition to the compounds of the present invention. In a further aspect, the additional pharmacological agent or agents may be co-administered, either simultaneously or sequentially, with the pharmaceutical compositions of the invention.

The composition of the invention will normally be administered to humans such that the daily dose will be 0.01 to 75 mg/kg body weight and preferably 0.1 to 15 mg/kg body weight. A preferred composition of the invention is one suitable for oral administration in unit dosage form for example a tablet or capsule which contains from 1 to 1000 mg and preferably 10 to 500 mg of a compound according to the present invention in each unit dose.

Thus, according to yet another aspect of the invention, there is provided a compound of formulae (I), (II), (II) or (IV) or a pharmaceutically acceptable salt or an in vivo hydrolysable derivative thereof for use in a method of therapeutic treatment of the human or animal body.

In yet a further aspect of the invention the present invention provides a method of treating a disease mediated by the interaction between VCAM-1 and/or fibronectin and the integrin receptor α₄β₁ in need of such treatment which comprises administering to said warm-blooded mammals an effective amount of a compound of formulae (I), (II), (III) or (IV) or a pharmaceutically acceptable salt or an in vivo hydrolysable derivative thereof.

The present invention also provides the use of a compound of formulae (I), (II), (III) or (IV) or a pharmaceutically acceptable salt or an in vivo hydrolysable derivative thereof in the production of a medicament for use in the treatment of a disease or medical condition mediated by the interaction between fibronectin and/or VCAM-1 (especially VCAM-1) and the integrin receptor α₄β₁.

In a preferred embodiment the mammal in need of treatment is suffering from multiple sclerosis, rheumatoid arthritis, asthma, coronary artery disease, psoriasis, atherosclerosis, transplant rejection, inflammatory bowel disease, insulin-dependent diabetes and glomerulonephritis.

In another aspect of the invention, there is provided a process for preparing a compound of formula (I), a pharmaceutically acceptable salt or an in vivo hydrolysable derivative thereof which process comprises coupling together a compound of formula (VI)

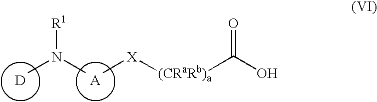

where D, A, R¹, X, R^{a}, R^{b} and a are as defined hereinbefore in relation to formula (I) and an appropriate amine of formula (VII)

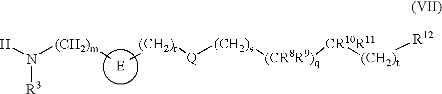

where R³, E, Q, R⁸, R⁹, R¹⁰, R¹¹, R¹², m, r, s, q and t are as hereinbefore defined in relation to formula (I) provided that any functional group is optionally protected; and thereafter, if necessary:

a) removing any protecting group; and

b) forming a pharmaceutically acceptable salt or in vivo hydrolysable derivative.

The reactions to couple the acids of formula (VI) to the amines of formula (VII) are suitably performed under standard coupling conditions for forming peptide bonds. They can be performed either on a solid support (Solid Phase Peptide Synthesis) or in solution using normal techniques used in the synthesis of organic compounds. With the exception of the solid support, all the other protecting groups, coupling agents, deblocking reagents and purification techniques are similar in both the solid phase and solution phase peptide synthesis techniques.

During the reaction, amino acid functional groups may, if necessary, be protected by protecting groups, for example BOC (tert-butoxycarbonyl). Such groups can be cleaved when necessary using standard techniques such as acid or base treatment.

Suitable protecting groups for the protection of the carboxyl groups include esters.

Coupling reagents for forming peptide bonds include the commonly used azide, symmetrical anhydride, mixed anhydride and various active esters and carbodiimides. In the case of carbodiimides, additives such as 1-hydroxybenzotriazole in particular N-hydroxybenzotriazole hydrate (HOBT) and N-hydroxysuccinimide may also be added. Other coupling reagents include 1H-benzotriazole-1-yl-oxy-tris-pyrrolidinophosphonium hexafluorophosphate (PyBOP), (2-(1H-benzotriazole-1-yl)-1,1,3,3-tetramethyluronium tetrafluoroborate (TBTU), (2-(1H-benzotriazole-1-yl)-1,1,3,3-tetramethyluronium hexafluorophosphate (HBTU)] and O-(7-azabenzotriazol-1-yl)-1,1,3,3-tetramethyluronium hexafluorophosphate (HATU).

The coupling reactions can be performed at temperatures between −20° C. to 40° C. The time of the reaction can vary such as between 10 minutes and 24 hours.

Suitable purification methods for the intermediates and final products include chromatographic techniques such as high pressure liquid chromatography (HPLC) along with many other standard techniques used in organic chemistry (e.g. solvent extraction and crystallisation).

Compounds of formula (VI) and (VII) may be prepared by conventional methods. For example, compounds of formula (VI), where A is benzoxazolyl, D is phenyl and R¹ is hydrogen may be prepared by cyclisation of a compound of formula (XV) using conventional methods. Compounds of formula (XV) which themselves may be prepared from compounds of formula (XII) by way of a compound of formula (XIV). In the following formula, R⁴¹ to R⁴³ are possible substituents on the bicyclic ring system A as hereinbefore defined, and R⁴¹ is preferably a group R⁴⁰ as defined above.

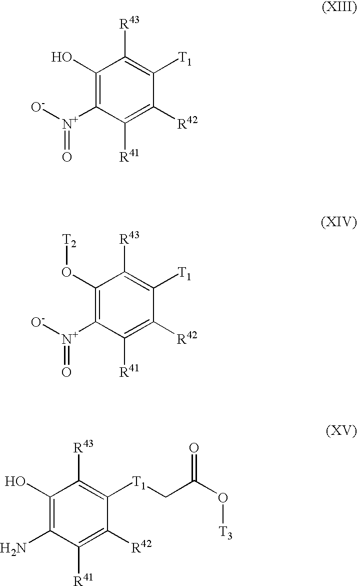

o-Nitrophenols of the type (formula (XIII) T₁=CH₂.CO₂Me) can be prepared by a variety of methods which include displacement of fluorine in compounds (formula (XIII) T₁=F) by diethyl sodiomalonate followed by hydrolysis and decarboxylation; and Pd mediated coupling with diethyl malonate of the compound (formula (XII) T₁=Br and where the hydroxy is preferably protected). Displacement of the fluorine in compounds of the type (formula (XIV); T₁=F, T₂=Bn) with hydroxide ion gives phenols (formula XIV) T₁=OH T₂=Bn) which can be reacted under basic conditions with t-butylbromoacetate to give t-butyl phenoxyacetates ((formula (XIV) T₁=OCH2CO.O^{t}Bu, T₂=Bn). The benzyl protecting group can be removed (e.g. Pd/H₂, Pd/ammonium formate or BBr₃) to yield a nitro phenol ((formula (XIV) T₁=OCH₂CO.O^{t}Bu, T₂=H). O-nitrophenols of the type (formula (XIV) T₁=CH₂.CO₂Me, T₂=Bn) can be prepared by Pd mediated coupling with diethyl malonate of the compound (formula (XIV) T₁=Br, T₂=Bn). The benzyl protecting group can be removed as described above.

Nitro phenols prepared as above can be reduced to an amino compound (formula (XIV) T₁=oxygen or direct bond, T₃=Me or ^{t}Bu) using, for example Pd/H₂, Pd/ammonium formate or Fe/HOAc. The amino compounds (formula(XV)) are unstable and can be converted in situ into the corresponding alkyl 2-phenylaminobenzoxazole-6-acetate (formula (XVI) T₁=O or direct bond, T₃=Me, ^{t}Bu) using an appropriately

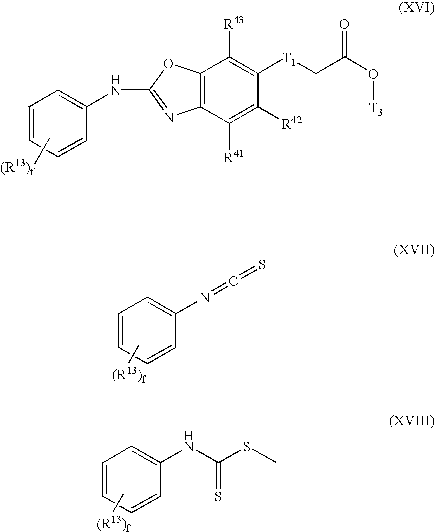

substituted phenyl isothiocyanate (XVII) or with an appropriately substituted phenyl dithiocarbamate (XVIII) in the presence of mercuric oxide. Deprotection of these esters will yield the corresponding acids ((formula (XVI), T₃=H).

An alternative route for the preparation of anilinobenzoxazoles and which avoids the need to use toxic mercuric oxide involves reacting o-hydroxyureas using Mitsunobu reaction conditions, i.e a trisubstituted triphosphine, for example tributylphosphine or triphenylphosphine and an azodicarbonyl compound, for example 1,1′-(azodicarbonyl)dipiperidine (ADDP) or diethylazodicarboxylate. This reaction can be carried out under mild conditions, is tolerant of a wide range of functional groups, is reliably reproducible and avoids the problem of handling and disposing of toxic reagents and residues. It also eliminates the potential for contaminating the final product with traces of mercury compounds.

Starting from compounds of formula (XIII), T₁=CO₂H and using similar methods, anilinobenzoxazoles acids of formula (XIX), T₃=CO₂H may be prepared.

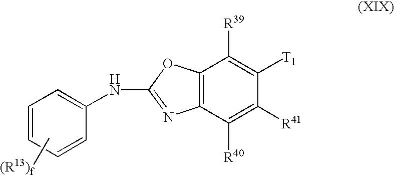

It will be understood that all amino acids are the natural isomers unless otherwise stated.

The invention is further limited by the following biological test methods, data and non-limiting examples, as described below and with reference to Table 1.

In the following examples:

¹H NMR data is quoted and is in the form of delta values for major diagnostic protons, given in parts per million (ppm) relative to tetramethylsilane (TMS) as an internal standard;

nitrogen atoms which are shown as less than trivalent are H substituted to complete the trivalency;

the following abbreviations are used:

DMSO dimethyl sulphoxide;

DMF N,N-dimethylformamide;

DCM dichloromethane;

DIPEA N,N-diisopropylethylamine;

EtOAc ethyl acetate;

HOBT N-hydroxybenzotriazole hydrate

HATU O(7-azabenzotriazol-1-yl)-N,N,N,′N′-tetramethyluronium hexafluorophosphate;

NMM N-Methylmorpholine;

TFA Trifluoroacetic acid;

WSCDI 1-(3-dimethylaminopropyl)-3-ethylcarbodiimide hydrochloride.

Compounds were purified by chromatography on Biotage Flash 40 KP-SIL silica 32-63 μm, 60A⁰ columns (8 g, 40 g, 90 g as appropriate for the amount of material to be purified).

### EXAMPLE 1

Preparation of Compound CVI in Table 1

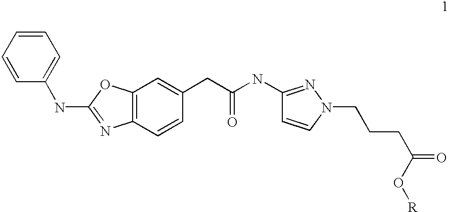

The methyl ester 1 R=Me, (324 mg, 0.75 mmol) in DMSO (2.0 ml) under an argon atmosphere was treated with aqueous NaOH (0.75 ml, 2M solution) and left to stir overnight. The solution was diluted with water (4 ml), the cloudy mixture extracted with ether (2×5 ml) and the mother liquors filtered through an ‘Acrodisc’ (0.45 μm PTFE) before adjusting to PH˜4 with 4M HCl. The title compound (1 where R═H) was obtained as a white solid (93 mg, 30% yield) following centrifugation, washing with H₂O, MeOH, and Et₂O and drying under vacuum

¹H NMR (DMSO d₆, 400 MHz) δ: 1.94 (2H, m); 2.15 (2H, t); 3.63 (2H, s); 3.99, 2H, s); 6.41 (1H, d); 7.02 (1H, t); 7.16 (H, dd); 7.34 (3H, t); 7.43 (1H, d); 7.53 (1H, d); 7.73 (1H, d); 7.75 (1H, d); 10.56 (1H, s); 10.62 (1H, s); 12.2 (1H, bs). LC/MS (ES⁺) m/z 420 (MH⁺) 99% pure; (ES⁻) 420 (MH⁻).

Step 1A

Preparation of Methyl Ester, 1,

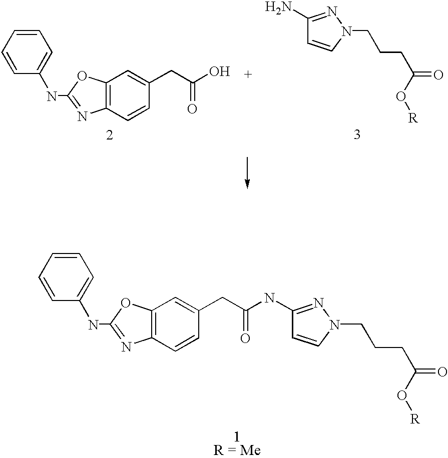

A mixture of the acid 2 (536 mg, 2 mmol), the amine 3 (366 mg, 2 mmol), WSCDI (575 mg, 3 mmol), HOBT (405 mg, 3 mmol) and N-methyl morpholine (1.1 ml, 10 mmol) in dry DMF (7.5 ml) under an atmosphere of argon, was stirred at room temp. overnight. The DMF was removed under reduced pressure, the residue partitioned between EtOAc and H₂O, and the organic extracts washed with H₂O and with brine before drying (Na₂ SO₄) and evaporation to give an oil (1.4 g) which partially solidified. The CH₂ Cl₂-soluble portion of this oil was purified by chromatography on silica (40 g) by eluting with EtOAc/iso-Hexane (3/2). Appropriate fractions (identified by tlc on silica) were combined and evaporated to give the ester 1, R=Me (564 mg, 65%) as a white solid.

¹H NMR (CDCl₃, 400 MHz) δ: 1.98 (2H, m); 2.22 (2H, t); 3.57 (2H, s); 3.62 (2H, s); 4.0 (2H, t); 6.4 (1H, d); 7.02 (1H, t); 7.17 (1H, dd); 7.35 (3H, m); 7.43 (1H, d); 7.54 (1H, d); 7.73, 7.77 (2H, dd); 10.55 (1H, s); 10.6 (1H, s).

MS (ES⁺) m/z m/z 434 (MH⁺).

Step 1b

Preparation of the Amine, 3.

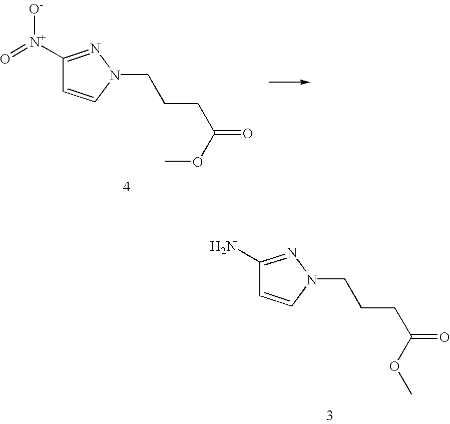

The nitropyrazole 4 (1.66 g, 7.7 mmol) in EtOH (10 ml) was treated with Pd/C (230 mg, 10%) and the mixture stirred in an atmosphere of hydrogen overnight. The catalyst was removed by filtration, washed with EtOH, and the solution evaporated to give 3 (1.38 g, 96.5%) as a colourless oil.

¹H NMR (CDCl₃, 500 MHz) δ: 2.13 (2H, m); 2.32 (2H, t); 3.59 (2H, bs); 3.67 (3H, s); 3.96 (2H, t); 5.56 (1H, d); 7.12 (1H, s).

MS (ES⁺) m/z 184 (MH⁺), 206 (M+Na⁺).

Step 1c

Preparation of the Nitropyrazole 4.

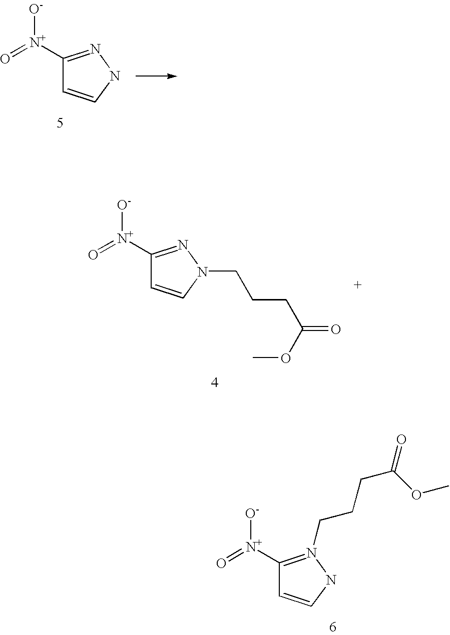

3-Nitropyrazole (1.13 g, 10 mmol) in dry DMF (10 ml), under argon, was treated with anhydrous K₂CO₃ (2.7 g, 15 mmol) and then, dropwise, with methyl-4-bromobutyrate (1.82 g, 10.05 mmol) at RT. After stirring overnight the DMF was removed and the residue partitioned between H₂O and EtOAc. The combined organic extracts were washed (H₂O and brine), dried (Na₂SO₄), filtered and evaporated to give a mixture of 4 and 6 (2.05 g) as a pale yellow oil.

MS (ES⁺) m/z 214 (MH⁺).

The individual isomers (1.66 g of 4, 77.9%; 318 mg of 6, 14.9%) were obtained following chromatography on silica (40 g) eluting with iso-hexane/EtOAc (7/3).

¹H NMR of 4 (CDCl₃, 400 MHz) δ: 2.26 (2H, m); 2.45 (2H, t); 3.68 (3H, s); 4.28 (2H, t); 6.9 (1H, d); 7.48 (1H, d).

The NMR assignments for 4 were underpinned by nOe experiments.

¹H NMR of 6 (CDCl₃, 400 MHz) δ: 2.24 (2H, m); 2.49 (2H, t); 3.68 (3H, s); 4.68 (2H, t); 7.06 (1H, d); 7.5 (1H, d)

### EXAMPLE 2

The Preparation of Compound CVII in Table 1

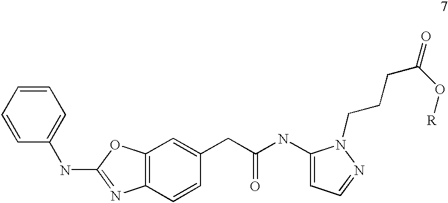

The title compound CVII in Table 1 (7 where R═H) was prepared from the methyl ester 7, R=Me, by the same procedure as used in the Example 1 but in this instance the product contained 27% unreacted 7, R=Me.

¹H NMR of Compound CVII in Table 1(DMSO d₆, 400 MHz) δ: 1.9 (2H, m); 2.12 (2H, t); 3.78 (2H, s); 4.02 (2H, t); 6.21 (1H, d); 7.04 (1H, t); 7.2 (1H, d); 7.32-7.42 (4H, m); 7.46 (1H); 7.76 (2H); 10.05 (1H, s); 10.6 (1H, s); 12.12 (1H, bs).

MS (ES⁺) m/z 420 (MH⁺).

Step 2A

Preparation of Methyl Ester

The methyl ester 7 R=Me, was prepared from the nitropyrazole 6 by following the same procedure as used in the conversion of the isomer 4 to 1, R=Me. Thus, the nitro compound 6 was reduced over Pd/C and the resulting amine 8 coupled to the acid 2 to give 7, R=Me (36% yield).

¹H NMR of 7 R=Me, (DMSO d₆, 500 MHz) δ: 1.89 (2H, m); 2.18 (2H, t); 3.55 (3H, s); 3.74 (2H, s); 3.95 (2H, t); 6.16 (1H, d); 7.02 (1H, t); 7.18 (1H, d); 7.3-7.38 (4H, m); 7.46 (1H, d); 7.74 (2H, d), 10.0 (1H, s); 10.58 (1H, s).

MS (ES⁺) m/z 434 (MH⁺).

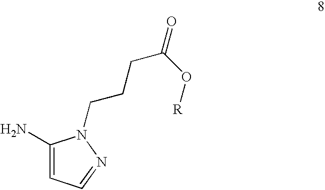

¹H NMR of 8 (CDCl₃, 300 MHz) δ: 2.14 (2H, m); 2.35 (2H, t); 3.68 (3H, s); 3.72 (2H, bs); 4.02 (2H, t); 5.51 (1H, d); 7.25 (1H, d).

MS of 8 (ES⁺) m/z 184 (MH⁺).

### EXAMPLE 3

Preparation of Compound CXVII in Table 1

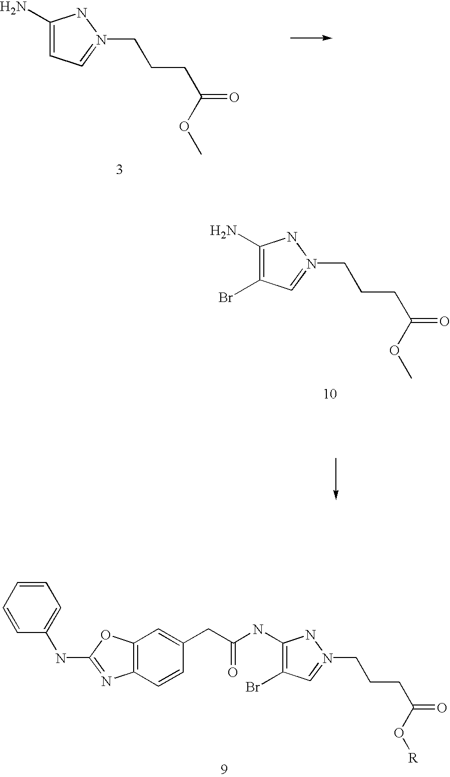

The methyl ester 9, R=Me, was hydrolysed to the title compound 17 in Table 1 (9 above where R═H) (54% yield) by hydrolysis with aqueous hydroxide in DMSO as described in Example 1.

¹H NMR of Compound CXVII (DMSO d₆, 400 MHz) δ: 1.98 (2H, m); 2.19 (2H, t); 3.67 (2H, s); 4.06 (2H, t); 7.02 (1H, t); 7.15(1H, m); 7.33-7.38 (3H, m); 7.42 (1H, bs); 7.72 (1H, d); 7.74 (1H, d); 7.85 (1H, s); 9.56 (1H, s); 10.32 (1H, s); 11.5-12.0 (1H, bs).

MS of Compound CXVII (ES⁺) m/z 498,500 (1×Br) (MH⁺).

Step 3a

The Preparation of 9 R=Me

A mixture of the acid 2 (352 mg, 1.3 mmol), the aminoester 10 (450 mg, 1.72 mmol), and HATU, (750 mg, 1.97 mmol) in dry DMF (5 ml) under argon, was treated with DIPEA (0.9 ml, 5.26 mmol) and stirred for 60 hr. The DMF was removed and the residue, in EtOAc, was washed with 4M HCl (3×5 ml), with aq. NaHCO₃ (2×5 ml), H₂O and brine before being dried, filtered and evaporated to give a foam (638 mg). The title compound was obtained—following chromatography on silica (40 g)—as solid (224 mg, 34%).

¹H NMR of 9 R=Me (CDCl₃, 400 MHz) δ: 2.0 (2H, m); 2.34 (2H, t); 3.6 (3H, s); 3.69 (2H, s); 4.04 (2H, t); 7.05 (1H, t); 7.18 (1H, d); 7.35-7.42 (3H, m); 7.44 (1H, s); 7.75 (2H, d); 7.92 (1H, s), 9.85 (1H, s); 10.6 (1H, s).

MS of 9 R=Me (ES⁺) m/z 511, 513 (1×Br)(MH⁺).

Step 3b

The Preparation of 10

The amino pyrazole 3 (345 mg, 1.89 mmol) in CHCl₃ (5 ml), under argon, was treated with Et₃N (0.32 ml, 2.27 mmol) and then dropwise, with Br₂ (0.11 ml, 2.08 mmol) and left to stir O/N. The mixture was diluted with CHCl₃, washed with H₂ 0 (2×), with brine and dried before being evaporated to give the title compound as an oil (454 mg, 92%).

¹H NMR (CDCl₃, 400 MHz) δ: 2.13 (2H, m); 2.34 (2H, t); 3.72 (3H, s); 3.73 (2H, s); 4.0 (2H, t); 7.2 (1H, s). NOe experiments established the identity of the product.

### EXAMPLE 4

The Preparation of Compound CVIII in Table 1

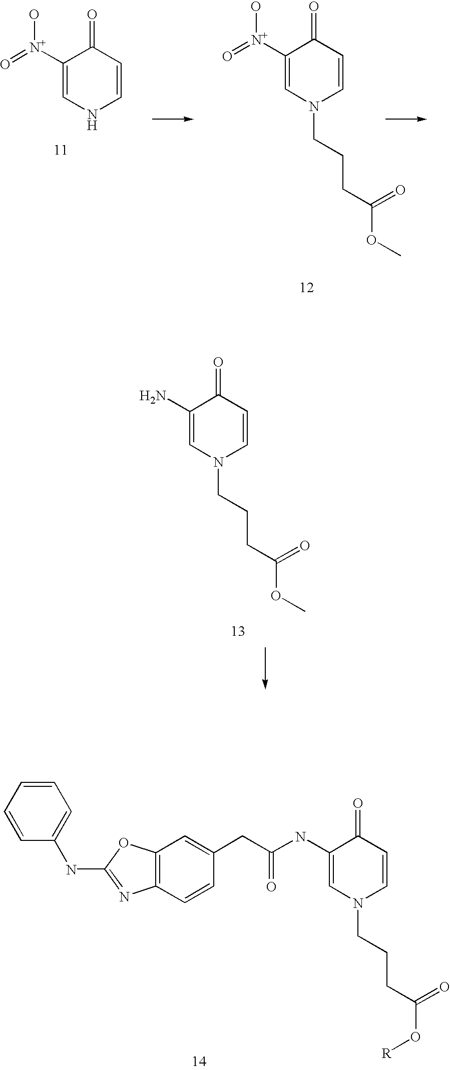

The methyl ester 14, R=Me, (102 mg, 0.22 mmol) in MeOH (3 ml) was heated on a steam bath with 2M NaOH (0.5 ml, 1 mmol) for 40 minutes. The solution was diluted with H₂O. (6 ml), the mixture extracted with Et₂O (2×), the pH of the aqueous mother liquors adjusted to 3 with 4M HCl and the title compound CVIII in Table 1 (14 where R═H) filtered off (57 mg, 92% pure, 57% yield).

¹H NMR of Compound CVIII in Table 1 (DMSO d₆, 500 MHz) δ: 1.88 (2H, m); 2.15 (2H, m); 3.85 (2H, s);3.9 (2H, t); 6.2 (1H, d); 7.02 (1H, t); 7.18 (1H, d); 7.3-7.4 (3H, m); 7.47 (1H, s); 7.65 (1H, dd); 7.74 (2H, d); 8.68 (1H, d); 9.16 (1H, s); 10.56 (1H, s); 12 (1H, bs).

MS of Compound CVII in Table 1 (ES⁺) m/z 447.

Step 4a

Preparation of 14, R=Me

The amine 13 (75 mg, 0.36 mmol), the acid 2 (115 mg, 0.43 mmol), WSCDI (103 mg, 0.53 mmol), HOBT (73 mg, 0.54 mmol) and NMM (0.2 ml, 1.82 mmol) in dry DMF (3 ml) under an atmosphere of argon was stirred at RT o/n. The DMF was removed under reduced pressure, the residue partitioned between EtOAc/H₂O, the combined extracts washed with H₂O, and with brine before drying over Na₂SO₄. Evaporation of the extracts gave a solid (134 mg) which when triturated with Et₂O gave the title compound (108 mg, 65% yield).

¹H NMR of 14, R=Me, (DMSO d₆, 400 MHz) δ: 1.96 (2H, m); 2.12 (2H, t); 3.57 (3H, s); 3.88 (2H, s); 3.94 (2H, t); 6.21 (1H, d); 7.0 (1H, t); 7.2 (1H, d); 7.35-7.42 (3H, m); 7.48 (1H, s); 7.66 (1H, d); 7.79 (2H, d); 8.70 (1H, d); 9.19 (1H, s); 10.6 (1H, s).

MS of 14, R=Me, (ES⁺) m/z 461 (MH⁺).

Step 4b

Preparation of 13

A solution of the nitropyridine 12 (100 mg, in EtOH (10 ml) was stirred in an atmosphere of hydrogen over 10%Pd/C (20 mg) until the uptake of H₂ was complete. The catalyst was removed by filtration through a pad of Celite, and the filtrate evaporated to give the title amine 13 (79 mg, 90% yield) as a solid.

¹H NMR of 13 (CDCl₃, 500 MHz) δ: 2.1 (2H, m); 2.34 (2H, t); 3.69 (3H, s); 3.84 (2H, t); 4.02 (2H, bs); 6.32 (1H, d); 6.92 (1H, s); 7.12 (1H, dd).

MS (ES⁺) m/z 211 (MH⁺).

Step 4c

Preparation of 12

4-Hydroxy-3-nitropyridine (500 mg, 3.6 mmol), was sired under argon with DMF (10 ml), and anhydrous K₂CO₃ (0.74 g, 5.4 mmol) until all the nitro-compound was in solution. The mixture was then treated dropwise at room temperature with methyl-4-bromobutyrate (0.64 g, 3.5 mmol) and stirred O/N.

The DMF was evaporated under vacuum, the residue partitioned between ETOAc and H₂O, extracted with EtOAc, and the combined extracts washed and dried as usual before being evaporated to give a solid (229 mg) which was triturated with Et₂O to give 12 as a solid (107 mg, 12% yield).

¹H NMR of 12 (CDCl₃, 400 MHz) δ: 2.2 (2H, m); 2.44 (2H, t); 3.73 (3H, s); 4.06 (2H, t); 6.66 (1H, d); 7.38 (1H, dd); 8.56 (1H, d).

NOe experiments confirmed that 12 is the N-alkylated isomer.

MS (ES⁺) m/z 241 (MH⁺).

### EXAMPLE 5

Preparation of Compound CXVIII in Table 1

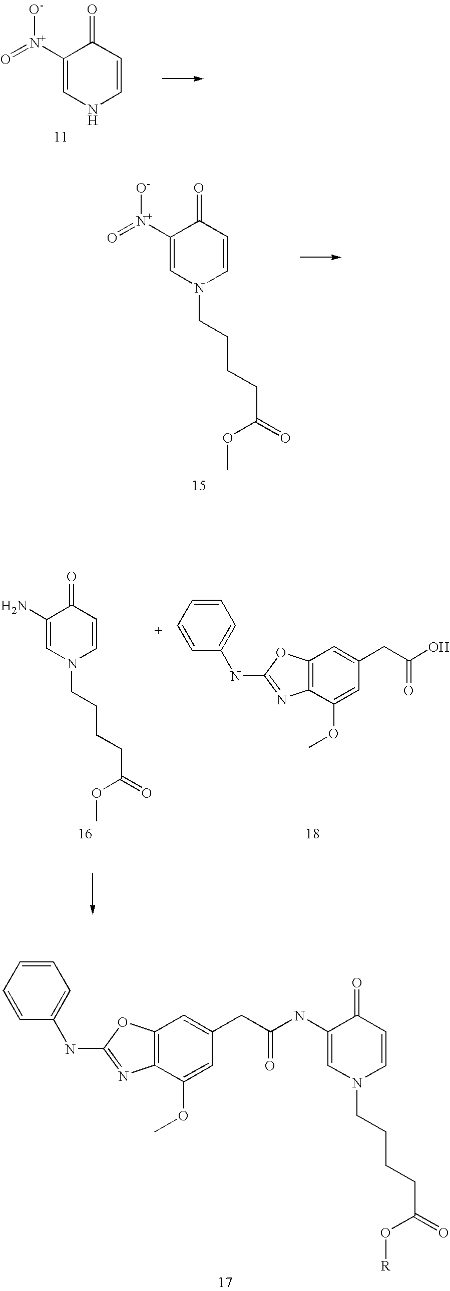

The title compound was obtained (in 20% yield) from the methyl ester 17, R=Me, following hydrolysis with aqueous NaOH/DMSO as described in Example 1.

¹H NMR of Compound CXVII in Table 1, (DMSO d₆, 400 MHz) δ: 1.45 (2H, m); 1.7 (2H, m); 2.24 (2H, t); 3.84 (2H, s); 3.92 (2H, t); 3.97 (3H, s); 6.22 (1H, d); 6.89 (1H, s); 7.03 (1H, t); 7.13 (1H, s); 7.37 (2H, t); 7.70 (1H, d); 7.75 (2H, d); 8.72 (1H, s); 9.2 (1H, s); 10.52 (1H, s); 12.02 (1H, bs).

MS of Compound CXVIII in Table 1, R═H, (ES⁺) m/z 491 (MH⁺).

Step 5a

Preparation of 17, R=Me

The title compound was prepared (in 50% yield) from the amine 16 and the methoxy acid 18 by the same coupling procedure as described for the preparation of 14 R=Me.

¹H NMR of 17, R=Me, (CDCl₃, 400 MHz) δ: 1.52 (2H, t); 1.71 (2H, t); 2.37 (2H, t); 3.58 (3H, s); 3.86 (2H, s); 3.96 (2H, t); 3.98 (3H, s); 6.21 (1H, d); 6.9 (1H, d); 7.03 (1H, t); 7.13 (1H, d); 7.38 (2H, t); 7.69 (1H, dd); 7.76 (2H, d); 8.72 (1H, d); 9.18 (1H, s); 10.52 (1H, s).

MS of 17, R=Me, (ES⁺) m/z 504 (MH⁺).

Step 5b

Preparation of 16

The amine 16 was obtained (in 88% yield) by reduction of the nitro compound 15 over 10% Pd/C as described in step 4b above.

¹H NMR of 16, (CDCl₃, 400 MHz) δ: 1.86 (2H, m); 1.7 (2H, m); 2.4 (2H, t); 3.72 (3H, s); 3.8 (2H, t); 4.04 (2H, bs); 6.34 (1H, d); 6.96 (1H, d); 7.15 (1H, dd).

MS (ES⁺) m/z 225 (MH⁺).

Step 5c

Preparation of 15

The nitro pyridone 15 was prepared (in 52% yield) from 4-Hydroxy-3-nitropyridine and methyl-5-bromovalerate by the same procedure as used in step 4c above for the preparation of 12.

¹H NMR of 15, (CDCl₃, 400 MHz) δ: 1.69 (2H, m); 1.92 (2H, m); 2.4 (2H, t); 3.69 (3H, s); 3.92 (2H, t); 6.69 (1H, d); 7.3 (1H, dd); 8.5 (1H, d).

The N-alkylated s was substantiated by ¹³C NMR (CDCl₃, 400 MHz) ppm: 21.3, 29.9, 32.9; 57.7; 138.0; 138.6; 141.7; 168.4.

The following scheme is to be used in conjunction with Examples 6-8 hereinafter.

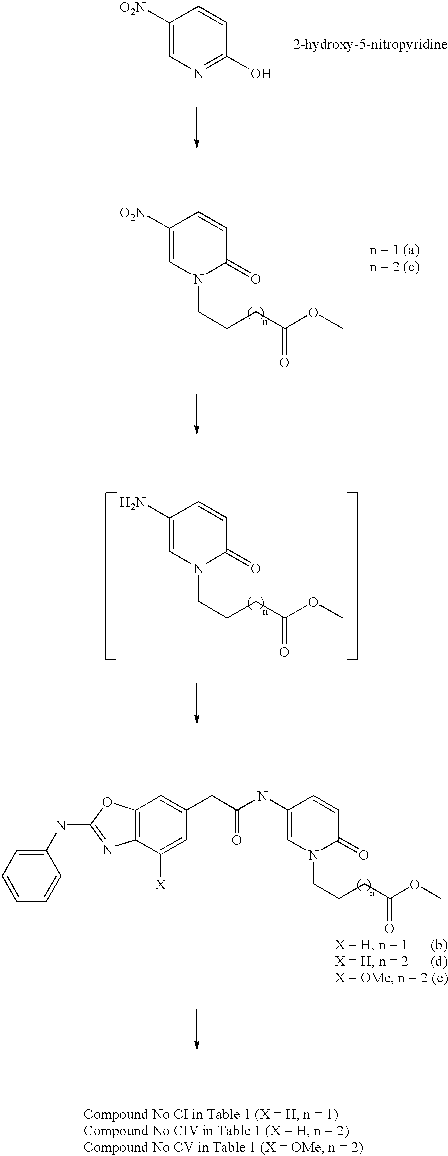

### EXAMPLE 6

Preparation of Compound CI in Table 1

A solution of (b) in the above scheme (0.115 g, 0.25 mmol) in DMSO (1.5 ml) was treated with 2M sodium hydroxide (0.35 ml, 0.7 mmol) and stirred for 3 days. The resulting mixture was then acidified with acetic acid and diluted with water. The precipitated product was collected by filtration, washed with water then diethyl ether and dried to give the title compound (0.104 g, 93%).

¹H NMR (DMSO d6, 300 MHz) d: 1.80 (2H, m); 2.20 (2H, t); 3.65 (2H, s); 3.84 (2H, t); 6.37 (1H, d); 7.00 (1H, t); 7.15 (1H, d); 7.30-7.44 (5H, m); 7.73 (2H, d); 8.05 (1H, d); 9.90 (1H, s).

MS (ES⁺) m/z 447 (MH⁺).

Step 6a

Preparation of (a)

A mixture of 2-hydroxy-5-nitropyridine (0.534 g, 3.81 mmol), methyl 4-bromobutyrate (0.725 g, 4.00 mmol), anhydrous potassium carbonate (2.11 g, 15.3 mmol) and DMF (7 mL) was stirred under a drying tube at room temperature overnight. The solvent was removed in vacuo, water (75 mL) was added and extracted with ethyl acetate (3×50 mL). The combined organic extracts were washed with brine (50 mL), dried MgSO₄), filtered and concentrated to dryness. The residue was purified by column chromatography (40 g Si) eluting with increasingly polar mixtures of methanol and DCM to give the title compound (a) as an orange oil (0.612 g, 67%).

¹H NMR (DMSO d6, 300 MHz) d: 1.94 (2H, m); 2.36 (2H, t); 3.55 (3H, s); 4.04 (2H, t); 6.48 (1H, d); 8.10 (1H, m); 9.11 (1H, d).

MS (ES⁺) m/z 241 (MH⁺).

Step 6b

Preparation of (b)

A mixture of (a) (0.154 g, 0.64 mmol), 10% Pd/C (0.02 g) and ethyl acetate (3 mL) was stirred under a hydrogen atmosphere for 4 hrs, then filtered to remove the catalyst and washed with ethylacetate (2×1 mL). To the resulting solution was added 2-phenylaminobenzoxazole-6-acetic acid (0.189 g, 0.71 mmol), HATU (0.293 g, 0.77 mmol), DIPEA (0.446 mL, 2.57 mmol) and DMF (3 mL). The mixture was placed under a drying tube and stirred at room temperature overnight. The solvents were removed in vacuo, ethyl acetate (100 mL) was added and the mixture was washed sequentially with water (30 mL), saturated sodium bicarbonate solution (3×25 mL) and brine (25 mL). The organic layer was dried (MgSO4), filtered, concentrated to dryness and purified by column chromatography (40 g Si) eluting with increasingly polar mixtures of methanol and DCM to give the title compound (b) as a brown solid (0.124 g, 42%).

¹H NMR (DMSO d6, 300 MHz) d: 1.84 (2H, m); 2.30 (2H, t); 3.35 (3H, s); 3.64 (2H, s); 3.95 (2H, t); 6.37 (1H, d); 7.01 (1H, t); 7.16 (1H, d); 7.30-7.43 (5H, m); 7.74 (2H, d); 8.04 (1H, d); 9.89 (1H, s); 1056 (1H, s)

MS (ES⁺) m/z 461 (MH⁺).

### EXAMPLE 7

Preparation of Compound CIV in Table 1

This was prepared by hydrolysis of (d) in the above scheme (0.120 g, 0.25 mmol) with 2M sodium hydroxide (0.303 mL, 0.61 mmol) over 6 hrs using the process described in Example 1. The acidified mixture was left to stand overnight before filtration. The title compound was obtained as a yellow solid (0.103 g, 88%).

¹H NMR (DMSO d6, 300 MHz) d: 1.39-1.53 (2H, m); 1.53-1.66 (2H, m); 2.21 (2H, t); 3.63 (2H, s); 3.82 (2H, t); 6.36 (1H, d); 7.01 (1H, t); 7.15 (1H, d); 7.29-7.46 (5H, m); 7.73 (2H, d); 8.04 (1H, d); 9.88 (1H, s); 10.56 (1H, s).

MS (ES⁺) m/z 461 (MH⁺).

### EXAMPLE 8

Preparation of Compound CV in Table 1

This was prepared by hydrolysis of (e) (0.128 g, 0.25 mmol) with 2M sodium hydroxide (0.404 mL, 0.81 mmol) overnight using the process described in Example 1. The title compound was obtained as a yellow solid (0.075 g, 60%).

¹H NMR (DMSO d6, 300 MHz) d: 1.40-1.52 (2H, m); 1.52-1.65 (2H, m); 2.11 (2H, t); 3.61 (2H, s); 3.81 (2H, t); 3.94 (3H, s); 6.36 (1H, d); 6.80 (1H, s); 7.00 (1H, t); 7.05 (1H, s); 7.30-7.42 (3H, m); 7.73 (2H, d); 8.05 (1H, d); 9.85 (1H, s); 10.49 (1H, s).

MS (ES⁺) m/z 491 (MH⁺).

Step 8a

Preparation of (c)

This was prepared by alkylation of 2-hydroxy-5-nitropyridine (1.01 g, 7.21 mmol) with methyl 5-bromovalerate (1.48 g, 7.57 mmol), anhydrous potassium carbonate (3.98 g, 28.9 mmol) and DMF (10 mL) using the process described in Example 1(b). The reaction was performed at 70° C. over 4 hrs and the title compound was obtained as a yellow oil (0.825 g, 45%).

¹H NMR (DMSO d6, 300 MHz) d: 1.53 (2H, m); 1.68 (2H, m); 2.34 (2H, t); 3.56 (3H, s); 4.00 (2H, t); 6.46 (1H, d); 8.10 (1H, m); 9.16 (1H, d).

MS (ES⁺) m/z 255 (MH⁺).

Step 8b

Preparation of (d) and (e)

A mixture of (c) (0.804 g, 3.17 mmol), 10% Pd/C (0.08 g) and ethyl acetate (10 mL) was stirred under a hydrogen atmosphere for 3 hrs, then filtered to remove the catalyst and washed with ethyl acetate (2×5 mL). The resulting solution was divided into two portions of equal volume and to each was added WSCDI (0.457 g, 2.37 mmol), 1-hydroxybenzotriazole (0.321 g, 2.37 mmol), NMM (0.524 mL, 4.75 mmol) and DMF (10 mL). 2-Phenylaminobenzoxazole-6-acetic acid (0.467 g, 1.74 mmol) was added to one mixture and 4-methoxy-2-phenylaminobenzoxazole-6-acetic acid (0.519 g, 1.74 mmol) was added to the other. Both reactions were placed under a drying tube and stirred at room temperature overnight. The solvents were removed in vacuo, and each reaction was subjected to the following work-up: ethyl acetate (150 mL) was added and the mixture was washed sequentially with water (75 mL), saturated sodium bicarbonate solution (3×75 mL), water (75 mL) and brine (75 mL). The organic phase was dried (MgSO₄), filtered, concentrated to dryness and purified by column chromatography (40 g Si) eluting with increasingly polar mixtures of methanol and DCM to give the title compound (d) as a brown solid (0.311 g, 41%), and compound (e) as a purple solid which was dissolved in a small volume of DCM and precipitated by addition of diethyl ether, collected by filtration and dried (0.564 g, 71%).

(d) ¹H NMR (DMSO d6, 300 MHz) d: 1.42-1.65 (4H, m); 2.31 (2H, t); 3.54 (3H, s); 3.63 (2H, s); 3.81 (2H, t); 6.36 (1H, d); 7.00 (1H, t); 7.13 (1H, d); 7.30-7.43 (5H, m); 7.74 (2H, d); 8.04 (1H, d); 9.87 (1H, s); 10.56 (1H, s).

MS (ES⁺) m/z 475 (MH⁺).

(e) ¹H NMR (DMSO d6, 300 MHz) d: 1.42-1.64 (4H, m); 2.30 (2H, t); 3.55 (3H, s); 3.62 (2H, s); 3.82 (2H, t); 3.95 (3H, s); 6.36 (1H, d); 6.80 (1H, s); 7.00 (1H, t); 7.05 (1H, s); 7.30-7.41 (3H, m); 7.73 (2H, d); 8.05 (1H, d); 9.83 (1H, s); 10.48 (1H, s).

MS (ES⁺) m/z 505 (MH⁺).

### EXAMPLE 9

Preparation of Compound CIX in Table 1

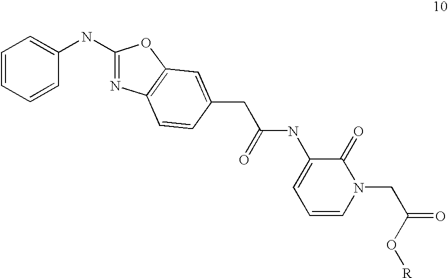

The methyl ester, 10 R=Me, (0.36 g, 0.833 mmol) was treated in MeOH (2 ml) with 2M NaOH (2.08 ml, 4.17 mmol) then heated at 60° C. for 15 mins. The resulting mixture was then diluted with water and acidified to pH 3 with 4M HCl. The precipitated product was filtered, washed with water and then with acetone to give the Compound CIX in Table 1 as a fawn solid (0.3 g, 0.718 mmol, Y=86%).

¹H NMR (DMSO d₆ 300 MHz): δ 3.84 (2H, s); 4.65 (2H, s); 6.23 (1H, t); 7.01 (1H, t); 7.17 (1H, d); 7.35 (4H, m); 7.46 (1H, s); 7.73 (2H, d); 8.20 (1H, d); 9.26 (1H, s); 10.56 (1H, s).

MS (ES⁺) m/z 419 (MH)⁺.

Step 9a)

Preparation of 10, R=Me

A mixture of methyl 3-nitropyrid-2-one-1-acetate (0.79 g, 3.73 mmol) in MeOH, 10% Pd/C (300 mg) and ammonium formate (0.52 g) was stirred for 24 hrs, the catalyst then removed by filtration and the filtrate evaporated to give an oil (0.68 g). Then a mixture of 2-phenylaminobenzoxazole-6-acetic acid (0.99 g, 3.69 mmol), HOBT (0.86 g, 5.62 mmol), WSCDI (1.07 g, 5.57 mmol), N-methylmorpholine (0.82 ml) in DMF (10 mil) was added and stirred for 48 hrs. Water added and the mixture extracted with EtOAc. The organic layer was separated, washed with water, 1M citric acid, aqueous NaHCO₃, brine, dried over MgSO₄ and evaporated to dryness to give a glass which when triturated with Et₂O and then with MeOH gave the product as a fawn solid (0.36 g, 0.83 mmol, Y=22%).

¹H NMR (DMSO d₆ 300 MHz) δ 3.65 (3H, s); 3.83 (2H, s); 4.75 (2H, s); 6.26 (1H, t); 7.01 (1H, t); 7.17 (1H, d); 7.34 (4H, m); 7.46 (1H, s); 7.73 (2H, d); 8.20 (1H, d); 9.28 (1H, s); 10.55 (1H, s).

MS (ES⁺) m/z 433 MH⁺.

Step 9b)

Preparation of Methyl 3-nitropyrid-2-one-1-acetate

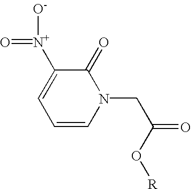

To a stirred suspension of NaH (60% oil dispersion) (0.314 g, 7.85 mmol) in DMF(1 ml) a solution of 2-hydroxy-3-nitropyridine(1 g, 7.14 mmol) in DMF(10 ml) was added dropwise, stirred for 1 hr, then a solution of methyl bromoacetate (1.15 g, 7.49 mmol) in DMF (2 ml) added and stirred for 18 hrs. EtOAc (50 ml) added, washed with 1M HCl, aqueous NaHCO₃, brine, dried over MgSO₄ and evaporated to give an oil which when triturated with isohexane and then Et 20 gave the product R=Me as a pink/fawn solid (0.79 g, 3.73 mmol, Y=52%)

¹H NMR (CDCl₃) δ 3.82 (3H, s); 4.77 (2H, s); 6.36 (1H, t); 7.64 (1H, d); 8.39 (1H, d).

MS (ES⁺) m/z 213 MH⁺.

### EXAMPLE 10

Preparation of Compound No CX in Table 1

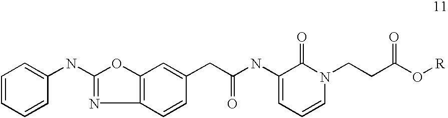

The title product was prepared by the hydrolysis of the methyl ester from 11, R=Me, using the process described in Example 9, to give the title product as a solid (0.31 g, 0.718 mmol, Y=80%).

¹H NMR (DMSO) δ 2.67 (2H, t); 3.85 (2H, s); 4.10 (2H, t); 6.21 (1H, t); 7.02 (1H, t); 7.30-7.40 (4H, m); 7.47 (1H, s); 7.74 (2H, d); 8.17 (1H, s); 9.26 (1H, s); 10.56 (1H, s); 12.36 (1H, s). Alkylation on nitrogen was confirmed by ¹³ C nmr.

MS (ES⁺) m/z 433 MH⁺.

Step 10a

Preparation of 11, R=Me

This was prepared by the process described in Example 9a but using methyl 3-nitropyrid-2-one-1-propionate to give the product as a solid (0.4 g, 0.897 mmol, Y=20%).

¹H NMR (DMSO d₆ 300 MHz) δ 2.76 (2H, t); 3.57 (3H, s); 3.85 (2H, s); 4.13 (2H, t); 6.21 (1H, t); 7.01 (1H, t); 7.19 (1H, d); 7.31-7.40 (4H, m); 7.47 (1H, s); 7.74 (2H, d); 8.18 (1H, d); 9.25 (1H, s); 10.56 (1H, s).

MS (ES⁺) m/z 447 (MH)⁺.

Step 10b

Preparation of Methyl 3-nitropyrid-2-one-1-propionate

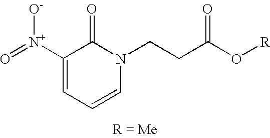

This was prepared by the process in Example 9b but using methyl 3-bromopropionate as the alkylating component to give the product as a oil (1.02 g, 4.25 mmol, Y=60%).

¹H NMR (CDCl₃) δ 2.95 (2H, t); 4.32 (2H, t); 6.30 (1H, t); 7.95 (1H, d); 8.35 (1H, d)

MS (ES⁺) m/z 227 (MH⁺).

### EXAMPLE 11

Preparation of Compound CXI in Table 1

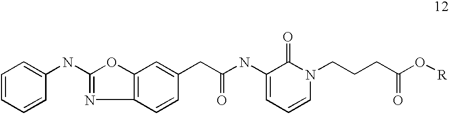

This was prepared by the hydrolysis of the methyl ester 12, R=Me, using the process described in Example 9 to give the title product as a solid (0.29 g, 0.65 mmol, 85.5%).

¹H NMR (DMSO d₆ 300 MHz) δ 1.86 (2H, m); 2.20 (2H, t); 3.84 (2H, s); 3.93 (2H, t); 6.22 (1H, t); 7.01 (1H, t); 7.18 (1H, d); 7.29-7.41 (4H, m); 7.47 (2H, d); 8.17 (1H, d); 9.24 (1H, s); 10.57 (1H, s)

MS (ES⁺) m/z 447 (MH⁺).

Step 11a

Preparation of 12, R=Me

This was prepared by the process described in step 9a above but using methyl 3-nitropyrid-2-one-1-butyrate to give the desired product as a solid (0.35 g, 0.76 mmol, Y=18%)

¹H NMR (DMSO d₆ 300 MHz) δ 1.91 (2H, m); 2.31 (2H, t); 3.84 (2H, s); 3.94 (2H, t); 6.22 (1H, t); 7.01 (1H, t); 7.18 (1H, d); 7.29-7.40 (4H, m); 7.47 (1H, s); 7.74 (2H, d); 8.17 (1H, d); 9.24 (1H, s); 10.57 (1H, s).

MS (ES⁺) m/z 461 MH⁺.

Step 11b

Preparation of Methyl 3-nitropyrid-2-one-1-butyrate

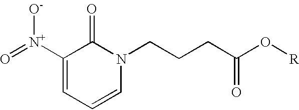

R=Me

This was prepared by the process in step 9b but using methyl 4-bromobutyrate as the alkylating component to give the product as an oil (0.99 g, 4.125 mmol, Y=58%).

¹H NMR (CDCl₃) δ 2.13 (2H, m); 2.42 (2H, t); 3.69 (3H, s); 4.15 (2H, t); 6.30 (1H, t); 7.71 (1H, d); 8.30 (1H, d).

MS (ES⁺) m/z 241 MH⁺.

### EXAMPLE 12

Preparation of Compound CXII in Table 1

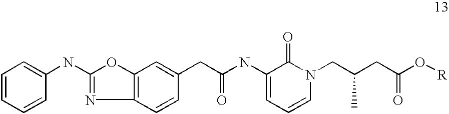

This was prepared by the hydrolysis of the ethyl ester from 13, R=Et using the process described in Example 9 but the product was purified by chromatography on silica (40 g) eluting with an increasingly polar mixture of MeOH/DCM and the appropriate fractions evaporated to yielded the title product as a glass (18 mg, 0.039 mmol, Y=20%).

1H NMR (DMSO d₆ 300 MHz) δ 0.91 (3H, d); 2.15 (2H, m); 3.85 (2H, s); 3.87 (2H, d); 6.20 (1H, t); 7.00 (1H, t); 7.10 (1H, d); 7.26 (1H, d); 7.29-7.38 (3H, m); 7.45 (1H, s); 7.75 (2H, d); 8.23 (1H, d); 9.20 (1H, s); 10.47 (1H, s).

MS (ES⁺) m/z 461 (MH⁺).

Step 12a)

Preparation of 13, R=Et

This was prepared by the process described in Step 9a but using

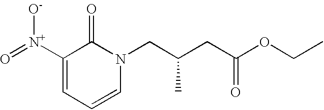

to give, after chromatography on silica (8 g cartridge) using an increasingly polar mixture of EtOAc/iso-hexane, the product as a glass (100 mg, 0.2 mmol, Y=30%).

¹H NMR (CDCl₃) δ 1.04 (3H, d); 1.26 (3H, t); 2.33 (2H, m); 2.58 (1H, m); 4.06 (2H, t); 4.13 (2H, q); 6.30 (1H, t); 7.74 (1H, m); 8.30 (1H, m).

MS (ES⁺) m/z 489 (MH⁺).

Step 12b)

Preparation of

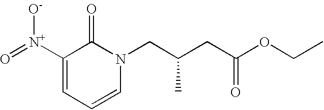

This was prepared by the process of Step 9b but using

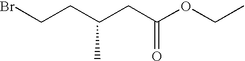

as the alkylating agent to give the desired product as an oil (0.18 g, 0.67 mmol, Y=29%).

¹H NMR (CDCl₃) δ 1.04 (3H, d); 1.26 (3H, t); 2.33 (2H, m); 2.58 (1H, m); 4.06 (2H, t); 4.13 (2H, q); 6.30 (1H, t); 7.74 (1H, m); 8.30 (1H, m).

MS (ES⁺) m/z 269 MH⁺.

### EXAMPLE 13

Preparation of Compound CXIII in Table 1

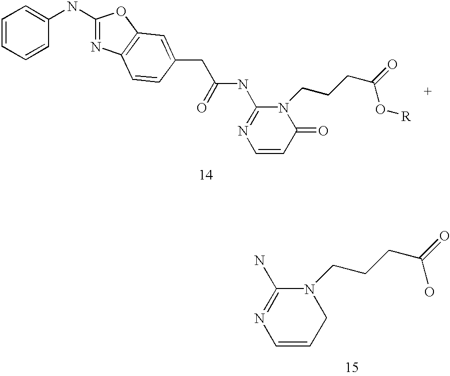

Note: 14 was contaminated with 15 (30%) because the amide was, in part, susceptible to the hydrolytic conditions.

The tBu ester 14 R=t-butyl (50 mg, 0.0994 mmol), Et3SiH (1 ml), DCM (3 ml) and TFA (3 ml) were stirred for 1 hr, evaporated to dryness and triturated with ether to give the product as a solid (16 mg, 0.036 mmol, Y=36%)

¹H NMR (DMSO d₆ 300 MHz) δ: 1.84 (2H, m); 2.23 (2H, t); 3.71 (2H, s); 3.90 (2H, t); 5.88 (1H, d); 7.00 (1H, t); 7.14 (1H, d); 7.34 (3H, t); 7.41 (1H, s); 7.73 (2H, d); 7.82 (1H, d).

MS (ES⁺) m/z 448 (MH⁺).

Step 13a)

Preparation of 14, R=t-Bu

A mixture of 16 (0.16 g, 4.4 mmol), K₂CO₃ (62 mg, 4.4 mmol), t-Butyl 4-bromobutyrate (99 mg, 4.4 mmol) and DMF was stirred and heated at 80° C. for 2 hrs. Cooled, water added and extracted with EtOAc. The organic extracts were washed with 1N NaOH, water, brine, dried over MgSO₄ and evaporated to dryness to give a gum. This was purified by chromatography on silica (Biotage KP Sil 8 g cartridge) eluting with an increasingly polar mixture of Et₂ O/isohexane and the appropriate fraction yielded the product (50 mg, 0.099 mmol, Y=23%)

¹H NMR (DMSO d₆ 300 MHz) δ 1.34 (9H, s); 1.78 (2H, m); 2.17 (2H, t); 3.70 (2H, s); 3.87 (2H, t); 5.88 (1H, d); 7.00 (1H, t); 7.14 (1H, d); 7.35 (3H, t); 7.40 (1H, s); 7.73 (2H, d); 7.80 (1H, d).

NMR correlation studies confirmed that the pyrimidone had been N-alkylated.

MS (ES⁺) m/z 504 MH⁺.

Step 13b

Preparation of

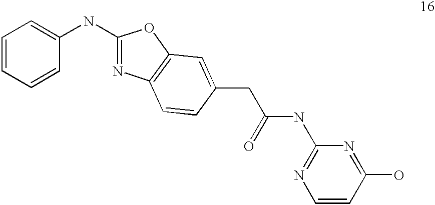

A mixture of 2-phenylaminobenzoxazole-6-acetic acid(0.48 g, 1.79 mmol), 2-amino-4-hydroxy pyrimidine(0.2 g, 1.79 mmol), WSCDI (0.52 g, 2.71 mmol), HOBT (0.41 g, 2.68 mmol), NMM (0.3 ml, 2.7 mmol) and DMF (10 ml) was stirs for 48 hrs. Water/aqueous NaHCO₃ (1:1) and EtOAc were added, the mixture stirred for 1 hr, and the product isolated as a solid (0.38 g, 1.05 mmol, Y=59%)

¹H NMR (DMSO d₆ 300 MHz) δ 3.82 (2H, s); 5.96 (1H, d); 7.00 (1H, t); 7.15 (1H, d); 7.35 (3H, m); 7.44 (1H, s); 7.73 (3H, m).

MS(ES⁺) m/z 362 MH⁺.

The following scheme relates to Examples 14 and 15

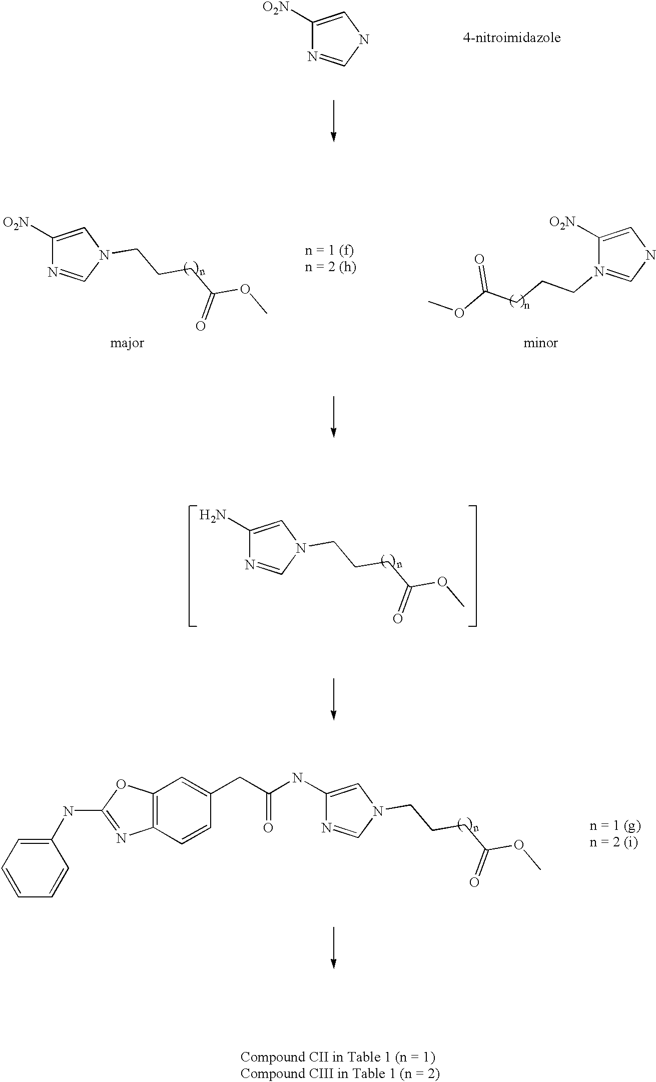

### EXAMPLE 14

Preparation of Compound CII in Table 1

This was prepared by hydrolysis of (g) in the above scheme (0.204 g, 0.47 mmol) with 2M sodium hydroxide (0.471 mL, 0.94 mmol) over 2 hrs using the process described in Example 1. During acidification the pH was adjusted to ˜6. The resulting precipitate was collected by filtration, washed with diethyl ether and dried to give the title compound as a yellow solid (0.082 g, 42%).

¹H NMR (DMSO d6, 300 MHz) d: 1.87(2H, m); 2.10 (2H, t); 3.63 (2H, s); 3.90 (2H, t); 7.00 (1H, t), 7.10-7.18 (2H, m); 7.29-7.44 (5H, m); 7.74 (2H, d), 10.42 (1H, s) 10.56 (1H, s).

MS (ES⁺) m/z 420 (MH⁺).

Step 14a

Preparation of (f)

This was prepared by alkylation of 4-nitroimidazole (1.0 g, 8.85 mmol) with methyl 4-bromobutyrate (1.76 g, 9.73 mmol), anhydrous potassium carbonate (2.44 g, 17.7 mmol) and DMF (15 mL) using the process described in Example 1 (b). The reaction was performed at 120° C. over 1.5 hrs and gave an orange oil (1.562 g, 83%). The title compound was obtained as a mixture of 1-alkylated (major) and 3-alkylated (minor) isomers (˜5:1), the NMR assignments were substantiated with nOe experiments.

¹H NMR (DMSO d6, 300 MHz) d: 1.96-2.08(2Hₘₐⱼ+2Hₘᵢₙ, m); 2.26-2.36 (2Hₘₐⱼ+2Hₘᵢₙ, m); 3.54 (3Hₘᵢₙ, s); 3.56 (3Hₘₐⱼ, s); 4.06 (2Hₘₐⱼ, t); 4.35 (2Hₘᵢₙ, t); 7.84 (1Hₘₐⱼ, s); 8.05 (2Hₘᵢₙ, s); 8.21 (1Hₘₐⱼ, s).

MS (ES⁺) m/z 214 (MH⁺).

Step 14b

Preparation of (a)

This was prepared from (f) (0.760 g, 3.57 mmol) using the process described in example 1(b). The reduction was performed with 10% Pd/C (0.075 g) in 1,4-dioxane (10 mL) and the filtered catalyst was washed with 1,4-dioxane (2×2 mL). The coupling reaction was performed with 2-phenylaminobenzoxazole-6-acetic acid (1.15 g, 4.28 mmol), HATU (1.63 g, 4.28 mmol), DIPEA (2.48 mL, 14.3 mmol) and DMF (10 mL). During work-up the organic layer was washed with 1M citric acid (3×75 mL) prior to the basic washes. Purification by column chromatography (90 g Si) eluting with increasingly polar mixtures of methanol and DCM gave the title compound (g) as a brown solid (0.579 g, 37%).

¹H NMR (DMSO d6, 300 MHz) d: 1.90 (2H, m); 2.22 (2H, t); 3.55 (3H, s); 3.63 (2H, s); 3.95 (2H, t); 7.00 (1H, t); 7.04 (1H, d); 7.06 (1H, s); 7.30-7.42 (5H, m); 7.73 (2H, d); 10.44 (1H, s); 10.56 (1H, s).

MS (ES⁺) m/z 434 (MH⁺).

### EXAMPLE 15

Preparation of Compound CIII

This was prepared by hydrolysis of (i) in the above scheme(0.112 g, 0.25 mmol) with 2M sodium hydroxide (0.251 mL, 0.50 mmol) over 1.5 hrs using the process described in Example 1. During acidification the pH was adjusted to 6. The resulting precipitate was then collected by filtration and washed with diethyl ether to give a yellow solid (0.087 g, 80%).

¹H NMR (DMSO d6, 300 MHz) d: 1.40(2H, m); 1.67 (2H, m); 2.20 (2H, t); 3.63 (2H, s); 3.88 (2H, t); 7.00 (1H, t); 7.10-7.20 (2H, m); 7.28-7.48 (5H, m); 7.75 (2H, d); 10.43 (1H, s); 10.55 (1H, s).

MS (ES⁺) m/z 434 (MH⁺).

Step 15a

Preparation of (h)

This was prepared by alkylation of 4-nitroimidazole (1.0 g, 8.85 mmol) with methyl 5-bromovalerate (1.90 g, 9.73 mmol), anhydrous potassium carbonate (2.44 g, 17.7 mmol) and DMF (15 mL) using the process described in step 1(b). The reaction was performed at 120° C. over 1.5 hrs and gave an orange oil (1.90 g, 95%). The title compound was obtained as a mixture of 1 alkylated (major) and 3-alkylated (minor) isomers (˜5:1), the NMR assignments were substantiated with nOe experiments.

¹H NMR (DMSO d6, 300 MHz) d: 1.40-1.62 (2Hₘₐⱼ+2H, m); 1.68-1.84 (2Hₘₐⱼ+2Hₘᵢₙ, m); 2.34 (2Hₘₐⱼ+2Hₘᵢₙ, t); 3.57 (3Hₘᵢₙ+3Hₘₐⱼ,s); 4.04 (2Hₘₐⱼ, t); 4.33 (2Hₘᵢₙ, t); 7.87 (1Hₘₐⱼ, s); 8.09 (1Hₘᵢₙ, s); 8.11 (1Hₘᵢₙ, s); 8.40 (1Hₘₐⱼ, s).

MS (ES⁺) m/z 228 (MH⁺).

Step 15b

Preparation of (i)

This was prepared from (h) in the above scheme (0.736 g, 3.24 mmol) using the process described in Step 1 (b). The reduction was performed with 10% Pd/C (0.074 g) in ethyl acetate (10 mL) and the filtered catalyst was washed with ethyl acetate (2×2 mL). The coupling reaction was performed with 2-phenylaminobenzoxazole-6-acetic acid (0.956 g, 3.57 mmol), HATU (1.54 g, 4.05 mmol), DIPEA (2.25 mL, 13.0 mmol) and DMF (15 mL). During work-up the organic layer was washed with 1M citric acid (3×50 mL) prior to the basic washes. Purification by column chromatography (40 g Si) eluting with increasingly polar mixtures of methanol and DCM gave the title compound (i) as a brown solid (0.464 g, 32%).

¹H NMR (DMSO d6, 300 MHz) d: 1.41 (2H, m); 1.65 (2H, m); 2.19 (2H, t); 3.47 (3H, s); 3.63 (2H, s); 3.98 (2H, t); 7.00 (1H, t); 7.11-7.18 (2H, m); 7.30-7.43 (5H, m); 7.73 (2H, d); 10.43 (1H, s); 10.55 (1H, s).

MS (ES⁺) m/z 448 (MH⁺).

### EXAMPLE 16

The compounds of the invention or pharmaceutically acceptable salts thereof may be formulated into tablets together with, for example, lactose Ph.Eur, Croscarmellose sodium, maize starch paste (5% w/v paste) and magnesium stearate for therapeutic or prophylactic use in humans. The tablets may be prepared by conventional procedures well known in the pharmaceutical art and may be film coated with typical coating materials such as hydroxypropylmethylcellulose.

In vitro and in vivo Assays

The following abbreviations are used. Suitable sources of materials are listed below.

MOLT4 cells—human T-lymphoblastic leukaemia cells European Collection of Anil Cell Cultures, Porton Down)

Fibronectin—purified from human plasma by gelatin-sepharose affinity chromatography according to the methods described in E. Nengvall, E. Ruoslahti, Int. J. Cancer, 1977, 20, pages 1-5 and J. Forsyth et al, Methods in Enzymology, 1992, 21 pages 311-316).

RPMI 1640—cell culture medium. (Life technologies, Paisley UK).

PBS—Dulbecco's phosphate buffered saline (Life Technologies).

BSA—Bovine serum albumin, fraction V (ICN, Thame, UK).

CrA—Complete Freund's Adjuvant (Life Technologies).

In the following assays and models references to compound(s) refers to the compounds of formula (I) and (II) according to the present invention.

1.1 In vitro Assay

1.1.1 MOLT-4 Cell/Fibronectin Adhesion Assay.

The MOLT-4 cell/fibronectin adhesion assay was used to investigate the interaction of the integrin α₄β₁ expressed on the MOLT-4 cell membrane with fibronectin. Polystyrene 96 well plates were coated overnight at 4° C. with fibronectin, 100 μl of 10 μg/ml in PBS. Non-specific adhesion sites were blocked by adding 100 μl BSA, 20 mg/ml. After incubating for 1 h at room temperature, the solutions were aspirated. MOLT-4 cells suspended in serum-free RPMI-1640 medium 2E6 cells/ml (50 μl) and solutions of compound diluted in the same medium (50 μl) were added to each well. After incubation for 2 h at 37° C. in a humidified atmosphere of 5% (v/v) CO₂, non-adherent cells were removed by gentle shaking followed by vacuum aspiration. Adherent cells were quantified by a colorimetric acid phosphatase assay. To each well was added 100 μl p-nitrophenyl phosphate (6 mg/ml) in 50 mM sodium acetate buffer, pH 5.0, containing 1% Triton X-100. After incubation for 1 h at 37° C., 50 μl sodium hydroxide (1M) was added to each well and the absorbance 405 nm was measured on a microplate spectrophotometer. Compounds which inhibited adhesion gave a lower absorbance reading. Standard, control and test conditions were assayed in triplicate. Percentage inhibition was calculated with respect to total (no inhibitor) and non-specific (no fibronectin) standards on each plate. Compounds of the invention were found to be active in this assay. For example Compound CV in Table 1 was an inhibitor at 1.1 μM.

1.2 In-vivo Inflammation Models

Activity of a compound can be tested in the following models.

1.2.1 Ovalbumin Delayed Type Hypersensitivity in Mice

Balb/c female mice (20-25 g) are immunised on the flank with an 1:1 (v/y) emulsion of ovalbumin (2 mg/ml) with CFk Seven days later the mice are challenged by subplantar injection of 1% heat aggregated ovalbumin in saline (30 μl) into the right bind foot pad. Swelling of the foot develops over a 24 hour period following which foot pad thickness is measured and compared with the thickness of the contralateral uninjected foot. The percentage increase in foot pad thickness is calculated. Compounds are dosed orally by gavage to groups of 5 mice at doses ranging from 0.001 mg/kg to 100 mg/kg. Inhibition of the inflammatory response is calculated comparing vehicle treated animals and compound treated groups.

1.2.2. Collagen-Induced Arthritis in Mice

DBA/1 male mice are immunised with 0.1 ml of an emulsion prepared from equal volumes of bovine collagen type II in 0.05M acetic acid (2 mg/ml) and CFA. This mixture is injected at the base of the tail. Twenty days later compounds are dosed orally by gavage at doses ranging from 0.001 mg/kg/day to 100 mg/kg/day. On the day following the first dose, each animal receives an intra-peritoneal booster injection of 0.1 ml of collagen type II in acetic acid. The mice are assessed for the incidence and severity of arthritis in all four limbs for up to 28 days. Inhibition of arthritis is calculated by comparing vehicle treated and compound treated mice.

Compounds of the invention are active in the above assays and screens.

1. A compound of formula (I) 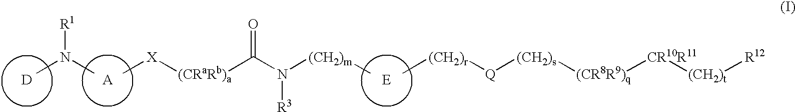
wherein:
A is a bicyclic heteroaryl group, optionally substituted with one or more substituents independently selected from C₁₋₆ alkyl, C₁₋₆ alkanoyl, C₂₋₆alkenyl, C₂₋₆alkynyl, C₁alkoxy, C₁₋₆ alkylamino, C₁₋₆ alkylthio, C₁₋₄ alkylsulphonyl, C₁₋₄alkoxylC₁₋₆alkyl, C₁₋₆alkylaminoC₁₋₆alkyl, carboxy, carbamoyl, C₂₋₆ alkenyloxy, C₂₋₆alkynyloxy, di-[(C₁₋₆)alkyl]amino, C₂₋₆alkanoylamino, N-C₁₋₆alkylcarbamoyl, C₁₋₆alkoxylcarbonyl, halogeno, nitro, cyano, amino trifluoromethyl, trifluoromethoxy, hydroxy, (CH₂)ₚOH where p is 1 or 2, —CO₂R^{e1}, and —CONR^{e1}R^{f1}, where R^{e1} and R^{f1} are independently hydrogen or C₁₋₆ alkyl; and linked to the nitrogen via a ring carbon atom in one ring and to the group Z by a ring carbon atom in the second ring;
D is aryl or a mono or bicyclic heteroaryl group, each of which can be optionally subsitituted with one or more substituents independently selected from C₁₋₆ alkyl, C₂₋₆alkenyl, C₂₋₆alkynyl, C₁₋₆ alkoxy, C₁₋₄ alkanoyl, C₁₋₆ alkylamino, C₁₋₆ alkylthio, C₁₋₄ alkylsulphonyl, C₁₋₄alkoxylC₁₋₆alkyl, C₁₋₆alkylaminoC₁₋₆alkyl, carboxy, carbamoyl, C₂₋₆ alkenyloxy, C₂₋₆alkynyloxy, di-[(C₁₋₆)alkyl]amino, C₂₋₆alkanoylamino, N-C₁₋₆alkylcarbamoyl, C₁₋₆alkoxylcarbonyl, phenoxy, cyano, nitro, amino, halogeno, trifluoromethyl, trifluoromethoxy, hydroxy, (CH₂)ₚOH where p is 1 or 2, —CO₂R^{e2}, and —CONR^{e2}R^{f2}, where R^{e2} and R^{f2} are as defined above, or two adjacent substitutents on the group D together with the ring atoms to which they are attached, form a 5-7membered optionally substituted ring which may contain up to three heteroatoms, and D is linked to NR¹ through a zing carbon atom;
R^{a} and R^{b} are independently hydrogen or C₁₋₄ alkyl;
a is an integer from 1 to 4;
X is a direct bond, oxygen, sulphur, amino or C₁₋₄alkylamino;
R¹ is hydrogen, C₁₋₅ alkyl, C₁₋₃ alkanoyl or C₁₋₃ alkoxycarbonyl;
R³ is hydrogen or C₁₋₅ alkyl;
E is a monocyclic or bicyclic heterocyclic ring containing at least one linking nitrogen atom, and which is optionally substituted with one or more substituents independently selected from oxo, C₁₋₆ alkyl, C₂₋₆alkenyl, C₂₋₆alkynyl, C₁₋₆ alkoxy, C₁₋₄ alkanoyl, C₁₋₆ alkylamino, C₁₋₄alkoxylC₁₋₆alkyl, C₁₋₆alkylaminoC₁₋₆alkyl, nitro, cyano, halogeno, trifluoromethyl, trifluoromethoxy, hydroxy, (CH₂)ₚOH where p is 1 or 2, —CO₂R^{e3}, and —CONR^{e3}R^{f3}, where R^{e3} and R^{f3} are independently selected from hydrogen and C₁₋₆ alkyl; and a substituent of formula (V)
—U—(CH₂)_{d}—V-T (V)
wherein U is selected from oxygen, sulphur, a direct bond or —CH₂O—, V is selected from nitrogen, oxygen, sulphur or a direct bond, d is zero or a number from 1 to 4, and T is selected from R^{c} or, when V is nitrogen, R^{c}R^{d},where R^{c} and R^{d} are independently selected from hydrogen, C₁₋₄ alkyl, C₁₋₄ alkoxy, C₁₋₄ alkoxy(C₁₋₆)alkyl or aryl; or T is a heterocycle containing up to three heteroatoms selected from nitrogen, oxygen and sulphur, optionally substituted with one or more substituents selected from C₁₋₆ alkyl, C₂₋₆alkenyl, C₂₋₆alkynyl, C₁₋₆ alkoxy, C₁₋₄ alkanoyl, C₁₋₆ alkylamino, C₁₋₄alkoxylC₁₋₆alkyl, C₁₋₆alkylaminoC₁₋₆alkyl, C₁₋₄ alkylsulphonyl, nitro, cyano, halogeno, trifluoromethyl, trifluoromethoxy, hydroxy, (CH₂)ₚOH where p is 1 or 2, —CO₂R^{e4}, and —CONR^{e4}R^{f4}, where R^{e4} and R^{f4} are independently selected from hydrogen and C₁₋₄ alkyl, and linked to V through a ring carbon or nitrogen and with the proviso that when T is a heterocycle linked to V through a ring nitrogen then V is a direct bond;
Q is selected from a direct bond, methylene, oxygen, carbonyl, —C(OH)(H)—, C₂ alkenyl or C₂ alkynyl;
R¹⁰ and each R⁸ and R⁹ are independently selected from hydrogen, C₁₋₆ alkyl, aryl and heterocycle, the aryl and heterocycle being optionally substituted with one or more substituents independently selected from C₁₋₆ alkyl, C₂₋₆alkenyl, C₁₋₄ alkanoyl, C₂₋₆alkynyl, C₁₋₆ alkoxy, C₁₋₆ alkylamino, C₁₋₄alkylC₁₋₆alkyoxyl, C₁₋₆alkylaminoC₁₋₆alkyl, nitro, cyano, halogeno, trifluoromethyl, hydroxy, (CH₂)ₚOH where p is 1 or 2, —CO₂R^{e5}, and —CONR^{e5}R^{f5}, where R^{e5} and R^{f5} are independently selected from hydrogen and C₁₋₆ alkyl, or two of R⁸, R⁹ and R¹⁰ together form a phenyl or a 3-7 membered heterocycle; R¹¹ is selected from hydrogen, C₁₋₆ alkyl, C₂₋₆alkenyl, 1,3-benzodioxol-5-yl, an ester group, hydroxy, amido, heterocycle and aryl, the heterocycle, and aryl optionally substituted with one or more substituents-independently selected from C₁₋₆ alkyl, C₂₋₆alkenyl, C₁₋₄alkanoyl, C₂₋₆alkynyl, C₁₋₆ alkoxy, C₁₋₆ alkylamino, C₁₋₄alkylC₁₋₆alkyoxyl, C₁₋₆alkylaminoC₁₋₆alkyl, nitro, cyano, halogeno, trifluoromethyl, hydroxy, (CH₂)ₚOH where p is 1 or 2, —CO₂R^{e6}, —CONR^{e6}R^{f6}, where R^{e6} and R^{f6} are independently selected from hydrogen and C₁₋₆ alkyl,
R¹² is an acidic functional group;
r is zero or 1;
q is 0, 1 or 2;
s is zero, 1 or 2;
t is zero or an integer of from 1 to 3;
m is zero or an integer of from 1 to 3;
or a pharmaceutically acceptable salt or in vivo hydrolysable derivative thereof.

2. A compound according to claim 1 wherein D is a phenyl optionally substituted with up to five substituents independently selected from C₁₋₆ alkyl, C₂₋₆alkenyl, C₂₋₆alkynyl, C₁₋₄ alkoxy, C₁₋₄ alkanoyl, C₁₋₆ alkylamino, C₁₋₄alkoxylC₁₋₆alkyl, C₁₋₆alkylaminoC₁₋₆alkyl, cyano, nitro, halogeno, trifluoromethyl, hydroxy, (CH₂)ₚOH where p is 1 or 2, are —CO₂R^{e}, and —CONR^{e2}R^{f2}, where R^{e2} and R^{f2} are independently hydrogen and C₁₋₆ alkyl, or two adjacent substituents can be taken together to form a 5-7 membered ring.

3. A compound according to claim 1 or claim 2 of formula (II) 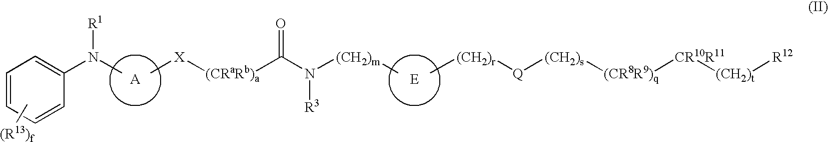
A, R¹, X, R^{a}, R^{b}, a, R³, E, m, r, Q, s, R⁸, R⁹, q, R¹⁰, R¹¹, t and and R¹² are as defined in claim 1;
each R¹³ is independently selected from C₁₋₆ alkyl, C₂₋₆alkenyl, C₂₋₆alkynyl, C₁₋₄ alkoxy, C₁₄ alkanoyl, C₁₋₆ alkylamino, C₁₋₄alkoxylC₁₋₆alkyl, C₁₋₆alkylaminoC₁₋₆alkyl, cyano, nitro, halogeno, trifluoromethyl, hydroxy, (CH₂)ₚOH where p is 1 or 2, —CO₂R^{e2}, and —CONR^{e2}R^{f2}, where R^{e2} and R^{f2} are independently hydrogen and C₁₋₆ alkyl, or where f is at least 2, two adjacent groups R¹³ can be taken together to form a 5-7 membered ring; and
f is zero or an integer from 1 to 5.

4. A compound according to claim 3 of formula (III) 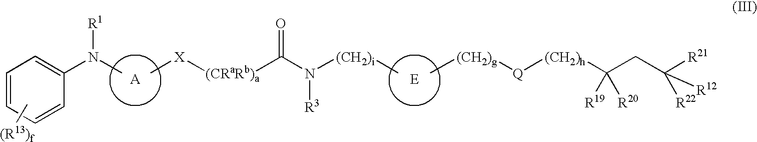
where A, R¹, Q, X, R^{a}, R^{b}, a, R³, E, R¹² are as defined in claim 1, R¹³ and f are as defined claim 3;
R¹⁹ to R²² are each independently selected from hydrogen, C₁₋₆ alkyl, aryl and heteroaryl containing up to 2 heteroatoms chosen from oxygen, sulphur and nitrogen, the aryl and heteroaryl optionally substituted with one or more substituents selected from nitro, C₁₋₆ alkyl, C₂₋₆alkenyl, C₂₋₆alkynyl, C₁₋₄ alkoxy, C₁₋₆ alkylamino, C₁₋₄alkylC₁₋₆alkyoxyl, C₁₋₆alkylaminoC₁₋₆alkyl, cyano, halogeno, trifluoromethyl, hydroxy, (CH₂)ₚOH where p is 1 or 2, —CO₂R^{e7}, and —CONR^{e7}R^{f7}, where R^{e7} and R_{f7} are independently selected from hydrogen and C₁₋₆alkyl or two of R¹⁹, R²⁰ or R²¹ can together form a phenyl or 3 to 7 membered heterocycle.
and g, h and i are each independently 0 or 1;
or a pharmaceutically acceptable salt or in vivo hydrolysable derivative thereof.

5. A compound according to claim 1 of formula ((IV) 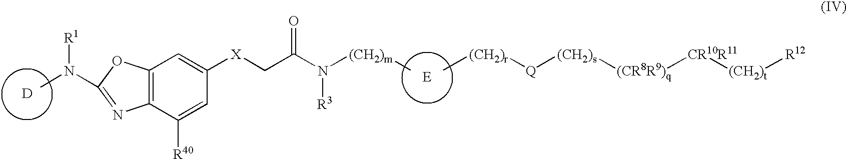
where
D, R¹, X, R³, E, Q, R⁸, R⁹, R¹⁰, R¹¹, R¹²,R¹², r, s, q and t are as defined in claim 1, and R⁴⁰ is hydrogen, C₁₋₄ alkoxy, halogeno, alkylthio and alkylsulphonyl.

6. A pharmaceutical composition which comprises a compound of formulae (I) as defined in claim 1, (II) as defined in claim 3, (III) as defined in claim 4 or (IV) as defined in claim 5 or a pharmaceutically acceptable salt or an in vivo hydrolysable derivative thereof and a pharmaceutically acceptable carrier.

7. A compound of formulae (I) as defined in claim 1, (II) as defined in claim 3, (III) as defined in claim 4 or (IV) as defined in claim 5 or a pharmaceutically acceptable salt or an in vivo hydrolysable derivative thereof for use in a method of therapeutic treatment of the human or animal body.

8. A method of treating a disease mediated by the interaction between VCAM-1 and/or fibronectin and the integrin receptor or α₄β₁ in need of such treatment which comprises administering to said warm-blooded mammals an effective amount of a compound of formulae (I) as defined in claim 1, (II) as defined in claim 3, (III) as defined in claim 4 or (IV) as defined in claim 5 or a pharmaceutically acceptable salt or an in vivo hydrolysable derivative thereof.

9. The use of a compound of formulae (I) as defined in claim 1, (II) as defined in claim 3, (III) as defined in claim 4 or (IV) as defined in claim 5 or a pharmaceutically acceptable salt or an in vivo hydrolysable derivative thereof in the production of a medicament for use in the treatment of a disease or medical condition mediated by the interaction between fibronectin and/or VCAM-1 and the integrin receptor α₄β₁.

10. A process for preparing a compound of formula (I) as defined in claim 1 or a pharmaceutically acceptable salt or an in vivo hydrolysable derivative thereof; which process comprises coupling together a compound of formula (VI) 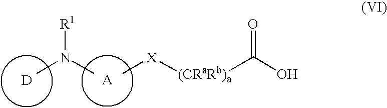
where D, A, R¹, X, R^{a}, R^{b} and a are as defined hereinbefore in relation to formula (I); and an amine of formula (VII) 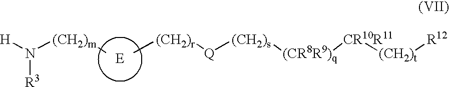
where R³, E, Q, R⁸, R⁹, R¹⁰, R¹¹, R¹², m, r, s, q and t are as defined in claim 1, provided that any functional group is optionally protected;
and thereafter, if necessary:
a) removing any protecting group; and
b) forming a pharmaceutically acceptable salt or in vivo hydrolysable derivative.
